SEQUENCE LISTING (28 sequences)

SEQ ID NO: 1 - DNA, 1983 bp, Homo sapiens
aggaacccagccatggtgaacgaagccagaggaaacagcagcctcaacccctgcttggagggcagtgccagcagtggcagtgagagctccaaagatagttcgagatgttccaccccgggcctggaccccgagcggcatgagagactccgggagaagatgaggcggcgattggaatctggtgacaagtggttctccctggaattcttccctcctcgaactgctgagggagctgtcaatctcatctcaaggtttgaccggatggcagcaggtggccccctctacatagacgtgacctggcacccagcaggtgaccctggctcagacaaggagacctcctccatgatgatcgccagcaccgccgtgaactactgtggcctggagaccatcctgcacatgacctgctgccgtcagcgcctggaggagatcacgggccatctgcacaaagctaagcagctgggcctgaagaacatcatggcgctgcggggagacccaataggtgaccagtgggaagaggaggagggaggcttcaactacgcagtggacctggtgaagcacatccgaagtgagtttggtgactactttgacatctgtgtggcaggttaccccaaaggccaccccgaagcagggagctttgaggctgacctgaagcacttgaaggagaaggtgtctgcgggagccgatttcatcatcacgcagcttttctttgaggctgacacattcttccgctttgtgaaggcatgcaccgacatgggcatcacttgccccatcgtccccgggatctttcccatccagggctaccactcccttcggcagcttgtgaagctgtccaagctggaggtgccacaggagatcaaggacgtgattgagccaatcaaagacaacgatgctgccatccgcaactatggcatcgagctggccgtgagcctgtgccaggagcttctggccagtggcttggtgccaggcctccacttctacaccctcaaccgcgagatggctaccacagaggtgctgaagcgcctggggatgtggactgaggaccccaggcgtcccctaccctgggctctcagcgcccaccccaagcgccgagaggaagatgtacgtcccatcttctgggcctccagaccaaagagttacatctaccgtacccaggagtgggacgagttccctaacggccgctggggcaattcctcttcccctgcctttggggagctgaaggactactacctcttctacctgaagagcaagtcccccaaggaggagctgctgaagatgtggggggaggagctgaccagtgaagaaagtgtctttgaagtcttcgttctttacctctcgggagaaccaaaccggaatggtcacaaagtgacttgcctgccctggaacgatgagcccctggcggctgagaccagcctgctgaaggaggagctgctgcgggtgaaccgccagggcatcctcaccatcaactcacagcccaacatcaacgggaagccgtcctccgaccccatcgtgggctggggccccagcgggggctatgtcttccagaaggcctacttagagtttttcacttcccgcgagacagcggaagcacttctgcaagtgctgaagaagtacgagctccgggttaattaccaccttgtcaatgtgaagggtgaaaacatcaccaatgcccctgaactgcagccgaatgctgtcacttggggcatcttccctgggcgagagatcatccagcccaccgtagtggatcccgtcagcttcatgttctggaaggacgaggcctttgccctgtggattgagcggtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacaggcccacccagaatgcgagagaaacggaggctccatga

SEQ ID NO: 2 - DNA, 3798 bp, Homo sapiens
atgtcacccgcgctccaagacctgtcgcaacccgaaggtctgaagaaaaccctgcgggatgagatcaatgccattctgcagaagaggattatggtgctggatggagggatggggaccatgatccagcgggagaagctaaacgaagaacacttccgaggtcaggaatttaaagatcatgccaggccgctgaaaggcaacaatgacattttaagtataactcagcctgatgtcatttaccaaatccataaggaatacttgctggctggggcagatatcattgaaacaaatacttttagcagcactagtattgcccaagctgactatggccttgaacacttggcctaccggatgaacatgtgctctgcaggagtggccagaaaagctgccgaggaggtaactctccagacaggaattaagaggtttgtggcaggggctctgggtccgactaataagacactctctgtgtccccatctgtggaaaggccggattataggaacatcacatttgatgagcttgttgaagcataccaagagcaggccaaaggacttctggatggcggggttgatatcttactcattgaaactatttttgatactgccaatgccaaggcagccttgtttgcactccaaaatctttttgaggagaaatatgctccccggcctatctttatttcagggacgatcgttgataaaagtgggcggactctttccggacagacaggagagggatttgtcatcagcgtgtctcatggagaaccactctgcattggattaaattgtgctttgggtgcagctgaaatgagaccttttattgaaataattggaaaatgtacaacagcctatgtcctctgttatcccaatgcaggtcttcccaacacctttggtgactatgatgaaacgccttctatgatggccaagcacctaaaggattttgctatggatggcttggtcaatatagttggaggatgctgtgggtcaacaccagatcatatcagggaaattgctgaagctgtgaaaaattgtaagcctagagttccacctgccactgcttttgaaggacatatgttactgtctggtctagagcccttcaggattggaccgtacaccaactttgttaacattggagagcgctgtaatgttgcaggatcaaggaagtttgctaaactcatcatggcaggaaactatgaagaagccttgtgtgttgccaaagtgcaggtggaaatgggagcccaggtgttggatgtcaacatggatgatggcatgctagatggtccaagtgcaatgaccagattttgcaacttaattgcttccgagccagacatcgcaaaggtacctttgtgcatcgactcctccaattttgctgtgattgaagctgggttaaagtgctgccaagggaagtgcattgtcaatagcattagtctgaaggaaggagaggacgacttcttggagaaggccaggaagattaaaaagtatggagctgctatggtggtcatggcttttgatgaagaaggacaggcaacagaaacagacacaaaaatcagagtgtgcacccgggcctaccatctgcttgtgaaaaaactgggctttaatccaaatgacattatttttgaccctaatatcctaaccattgggactggaatggaggaacacaacttgtatgccattaattttatccatgcaacaaaagtcattaaagaaacattacctggagccagaataagtggaggtctttccaacttgtccttctccttccgaggaatggaagccattcgagaagcaatgcatggggttttcctttaccatgcaatcaagtctggcatggacatggggatagtgaatgctggaaacctccctgtgtatgatgatatccataaggaacttctgcagctctgtgaagatctcatctggaataaagaccctgaggccactgagaagctcttacgttatgcccagactcaaggcacaggagggaagaaagtcattcagactgatgagtggagaaatggccctgtcgaagaacgccttgagtatgcccttgtgaagggcattgaaaaacatattattgaggatactgaggaagccaggttaaaccaaaaaaaatatccccgacctctcaatataattgaaggacccctgatgaatggaatgaaaattgttggtgatctttttggagctggaaaaatgtttctacctcaggttataaagtcagcccgggttatgaagaaggctgttggccaccttatccctttcatggaaaaagaaagagaagaaaccagagtgcttaacggcacagtagaagaagaggacccttaccagggcaccatcgtgctggccactgttaaaggcgacgtgcacgacataggcaagaacatagttggagtagtccttggctgcaataatttccgagttattgatttaggagtcatgactccatgtgataagatactgaaagctgctcttgaccacaaagcagatataattggcctgtcaggactcatcactccttccctggatgaaatgatttttgttgccaaggaaatggagagattagctataaggattccattgttgattggaggagcaaccacttcaaaaacccacacagcagttaaaatagctccgagatacagtgcacctgtaatccatgtcctggacgcgtccaagagtgtggtggtgtgttcccagctgttagatgaaaatctaaaggatgaatactttgaggaaatcatggaagaatatgaagatattagacaggaccattatgagtctctcaaggagaggagatacttacccttaagtcaagccagaaaaagtggtttccaaatggattggctgtctgaacctcacccagtgaagcccacgtttattgggacccaggtctttgaagactatgacctgcagaagctggtggactacattgactggaagcctttctttgatgtctggcagctccggggcaagtacccgaatcgaggctttcccaagatatttaacgacaaaacagtaggtggagaggccaggaaggtctacgatgatgcccacaatatgctgaacacactgattagtcaaaagaaactccgggcccggggtgtggttgggttctggccagcacagagtatccaagacgacattcacctgtacgcagaggctgctgtgccccaggctgcagagcccatagccaccttctatgggttaaggcaacaggctgagaaggactctgccagcacggagccatactactgcctctcagacttcatcgctcccttgcattctggcatccgtgactacctgggcctgtttgccgttgcctgctttggggtagaagagctgagcaaggcctatgaggatgatggtgacgactacagcagcatcatggtcaaggcgctgggggaccggctggcagaggcctttgcagaagagctccatgaaagagttcgccgagaactgtgggcctactgtggcagtgagcagctggacgtcgcagacctgcgcaggctgcggtacaagggcatccgcccggctcctggctaccccagccagcccgaccacaccgagaagctcaccatgtggagactcgcagacatcgagcagtctacaggcattaggttaacagaatcattagcaatggcacctgcttcagcagtctcaggcctctacttctccaatttgaagtccaaatattttgctgtggggaagatttccaaggatcaggttgaggattatgcattgaggaagaacatatctgtggctgaggttgagaaatggcttggacccattttgggatatgatacagactaa

SEQ ID NO: 3 - DNA, 2097 bp, Homo sapiens
atgaggaggtttctgttactatatgctacacagcagggacaggcaaaggccatcgcagaagaaatatgtgagcaagctgtggtacatggattttctgcagatcttcactgtattagtgaatccgataagtatgacctaaaaaccgaaacagctcctcttgttgttgtggtttctaccacgggcaccggagacccacccgacacagcccgcaagtttgttaaggaaatacagaaccaaacactgccggttgatttctttgctcacctgcggtatgggttactgggtctcggtgattcagaatacacctacttttgcaatggggggaagataattgataaacgacttcaagagcttggagcccggcatttctatgacactggacatgcagatgactgtgtaggtttagaacttgtggttgagccgtggattgctggactctggccagccctcagaaagcattttaggtcaagcagaggacaagaggagataagtggcgcactcccggtggcatcacctgcatcctcgaggacagaccttgtgaagtcagagctgctacacattgaatctcaagtcgagcttctgagattcgatgattcaggaagaaaggattctgaggttttgaagcaaaatgcagtgaacagcaaccaatccaatgttgtaattgaagactttgagtcctcacttacccgttcggtacccccactctcacaagcctctctgaatattcctggtttacccccagaatatttacaggtacatctgcaggagtctcttggccaggaggaaagccaagtatctgtgacttcagcagatccagtttttcaagtgccaatttcaaaggcagttcaacttactacgaatgatgccataaaaaccactctgctggtagaattggacatttcaaatacagacttttcctatcagcctggagatgccttcagcgtgatctgccctaacagtgattctgaggtacaaagcctactccaaagactgcagcttgaagataaaagagagcactgcgtccttttgaaaataaaggcagacacaaagaagaaaggagctaccttaccccagcatatacctgcgggatgttctctccagttcatttttacctggtgtcttgaaatccgagcaattcctaaaaaggcatttttgcgagcccttgtggactataccagtgacagtgctgaaaagcgcaggctacaggagctgtgcagtaaacaaggggcagccgattatagccgctttgtacgagatgcctgtgcctgcttgttggatctcctcctcgctttcccttcttgccagccaccactcagtctcctgctcgaacatcttcctaaacttcaacccagaccatattcgtgtgcaagctcaagtttatttcacccaggaaagctccattttgtcttcaacattgtggaatttctgtctactgccacaacagaggttctgcggaagggagtatgtacaggctggctggccttgttggttgcttcagttcttcagccaaacatacatgcatcccatgaagacagcgggaaagccctggctcctaagatatccatctctcctcgaacaacaaattctttccacttaccagatgacccctcaatccccatcataatggtgggtccaggaaccggcatagccccgtttattgggttcctacaacatagagagaaactccaagaacaacacccagatggaaattttggagcaatgtggttgttttttggctgcaggcataaggatagggattatctattcagaaaagagctcagacatttccttaagcatgggatcttaactcatctaaaggtttccttctcaagagatgctcctgttggggaggaggaagccccagcaaagtatgtgcaagacaacatccagcttcatggccagcaggtggcgagaatcctcctccaggagaacggccatatttatgtgtgtggagatgcaaagaatatggccaaggatgtacatgatgcccttgtgcaaataataagcaaagaggttggagttgaaaaactagaagcaatgaaaaccctggccactttaaaagaagaaaaacgctaccttcaggatatttggtcataa

SEQ ID NO: 4 - protein, 656 aa, Homo sapiens [sequence omitted]

SEQ ID NO: 5 - protein, 1265 aa, Homo sapiens
MSPALQDLSQPEGLKKTLRDEINAILQKRIMVLDGGMGTMIQREKLNEEHFRGQEFKDHARPLKGNNDILSITQPDVIYQIHKEYLLAGADIIETNTFSSTSIAQADYGLEHLAYRMNMCSAGVARKAAEEVTLQTGIKRFVAGALGPTNKTLSVSPSVERPDYRNITFDELVEAYQEQAKGLLDGGVDILLIETIFDTANAKAALFALQNLFEEKYAPRPIFISGTIVDKSGRTLSGQTGEGFVISVSHGEPLCIGLNCALGAAEMRPFIEIIGKCTTAYVLCYPNAGLPNTFGDYDETPSMMAKHLKDFAMDGLVNIVGGCCGSTPDHIREIAEAVKNCKPRVPPATAFEGHMLLSGLEPFRIGPYTNFVNIGERCNVAGSRKFAKLIMAGNYEEALCVAKVQVEMGAQVLDVNMDDGMLDGPSAMTRFCNLIASEPDIAKVPLCIDSSNFAVIEAGLKCCQGKCIVNSISLKEGEDDFLEKARKIKKYGAAMVVMAFDEEGQATETDTKIRVCTRAYHLLVKKLGFNPNDIIFDPNILTIGTGMEEHNLYAINFIHATKVIKETLPGARISGGLSNLSFSFRGMEAIREAMHGVFLYHAIKSGMDMGIVNAGNLPVYDDIHKELLQLCEDLIWNKDPEATEKLLRYAQTQGTGGKKVIQTDEWRNGPVEERLEYALVKGIEKHIIEDTEEARLNQKKYPRPLNIIEGPLMNGMKIVGDLFGAGKMFLPQVIKSARVMKKAVGHLIPFMEKEREETRVLNGTVEEEDPYQGTIVLATVKGDVHDIGKNIVGVVLGCNNFRVIDLGVMTPCDKILKAALDHKADIIGLSGLITPSLDEMIFVAKEMERLAIRIPLLIGGATTSKTHTAVKIAPRYSAPVIHVLDASKSVVVCSQLLDENLKDEYFEEIMEEYEDIRQDHYESLKERRYLPLSQARKSGFQMDWLSEPHPVKPTFIGTQVFEDYDLQKLVDYIDWKPFFDVWQLRGKYPNRGFPKIFNDKTVGGEARKVYDDAHNMLNTLISQKKLRARGVVGFWPAQSIQDDIHLYAEAAVPQAAEPIATFYGLRQQAEKDSASTEPYYCLSDFIAPLHSGIRDYLGLFAVACFGVEELSKAYEDDGDDYSSIMVKALGDRLAEAFAEELHERVRRELWAYCGSEQLDVADLRRLRYKGIRPAPGYPSQPDHTEKLTMWRLADIEQSTGIRLTESLAMAPASAVSGLYFSNLKSKYFAVGKISKDQVEDYALRKNISVAEVEKWLGPILGYDTD

SEQ ID NO: 6 - protein, 698 aa, Homo sapiens
MRRFLLLYATQQGQAKAIAEEICEQAVVHGFSADLHCISESDKYDLKTETAPLVVVVSTTGTGDPPDTARKFVKEIQNQTLPVDFFAHLRYGLLGLGDSEYTYFCNGGKIIDKRLQELGARHFYDTGHADDCVGLELVVEPWIAGLWPALRKHFRSSRGQEEISGALPVASPASSRTDLVKSELLHIESQVELLRFDDSGRKDSEVLKQNAVNSNQSNVVIEDFESSLTRSVPPLSQASLNIPGLPPEYLQVHLQESLGQEESQVSVTSADPVFQVPISKAVQLTTNDAIKTTLLVELDISNTDFSYQPGDAFSVICPNSDSEVQSLLQRLQLEDKREHCVLLKIKADTKKKGATLPQHIPAGCSLQFIFTWCLEIRAIPKKAFLRALVDYTSDSAEKRRLQELCSKQGAADYSRFVRDACACLLDLLLAFPSCQPPLSLLLEHLPKLQPRPYSCASSSLFHPGKLHFVFNIVEFLSTATTEVLRKGVCTGWLALLVASVLQPNIHASHEDSGKALAPKISISPRTTNSFHLPDDPSIPIIMVGPGTGIAPFIGFLQHREKLQEQHPDGNFGAMWLFFGCRHKDRDYLFRKELRHFLKHGILTHLKVSFSRDAPVGEEEAPAKYVQDNIQLHGQQVARILLQENGHIYVCGDAKNMAKDVHDALVQIISKEVGVEKLEAMKTLATLKEEKRYLQDIWS

SEQ ID NO: 7 - DNA, 52 bp, Homo sapiens
cttgaaggagaaggtgtctgcgggagycgatttcatcatcacgcagcttttc

SEQ ID NO: 8 - DNA, 52 bp, Homo sapiens
cgaggcctttgccctgtggattgagcrgtggggaaagctgtatgaggaggag

SEQ ID NO: 9 - DNA, 52 bp, Homo sapiens
ggaagaatatgaagatattagacaggrccattatgagtctctcaaggtaagt

SEQ ID NO: 10 - DNA, 52 bp, Homo sapiens
caggcaaaggccatcgcagaagaaatrtgtgagcaagctgtggtacatggat

SEQ ID NO: 11 - DNA, 52 bp, Homo sapiens
taagttccattccatctcagcccgaartgttttcagagccggagacctcaca

SEQ ID NO: 12 - DNA, 52 bp, Homo sapiens
ctgctgctggtatcagcctggaggaartgagtgacatcagttctcagcatta

SEQ ID NO: 13 - DNA, 52 bp, Homo sapiens
aaccaatcacaacaaggcagataaagwaggatgagttgtcagattttgataa

SEQ ID NO: 14 - DNA, 52 bp, Homo sapiens
gcttcggagctggagcgcatgaatccyggcccaggcgggaagctgggacacg

SEQ ID NO: 15 - DNA, 52 bp, Homo sapiens
aagctgagaacatcaagaagttcttayagtaagtacatcctcgaaagtttat

SEQ ID NO: 16 - DNA, 52 bp, Homo sapiens
gggagggcacccgcagaggcctgcgcrctgacactgctgagtggctctgctc

SEQ ID NO: 17 - DNA, 52 bp, Homo sapiens
tgaccccgagctccggtcctggcggcrcctcgtgtgctacctttgcttctac

SEQ ID NO: 18 - DNA, 52 bp, Homo sapiens
caataggagcgtgtgtttgaacagtayacgccaaacttcagtcattcaagta

SEQ ID NO: 19 - DNA, 52 bp, Homo sapiens
ggcctaatcaatccttctcatcttttrtacccaccttttgcaggaaacctgt

SEQ ID NO: 20 - DNA, 52 bp, Homo sapiens
ctgactctccccgacccgtcccaccayggtctccacagcactcccgacagcc

SEQ ID NO: 21 - DNA, 52 bp, Homo sapiens
gtccctgcagtttaattatcctcaacrttactgccataccctacatttttgg

SEQ ID NO: 22 - DNA, 52 bp, Homo sapiens
ctcacagtttgtggttgagactaagtrtgacaacagtggcactttgtggtcc

SEQ ID NO: 23 - DNA, 52 bp, Homo sapiens
accaaggagcagcgcatcctgaaccaygtgctgcagcatgcggagcccggga

SEQ ID NO: 24 - DNA, 52 bp, Homo sapiens
cccagcggatggtggatttcgctggcrtgaaggacaaggtgtgcatgcctga

SEQ ID NO: 25 - DNA, 52 bp, Homo sapiens
taatatggccaccccaactttcgtatsattactgtttgtgtggtattatctt

SEQ ID NO: 26 - DNA, 52 bp, Homo sapiens
atcagccctagatgcttgaccagctcytcgggcctcacctcctggttcttcc

SEQ ID NO: 27 - DNA, 52 bp, Homo sapiens
ctgggccaacaagcttgagtgcgatcyggtctgcaatgatggaggaattgcc

SEQ ID NO: 28 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

(19) United States
(12) Patent Application Publication
(10) Pub. No.: US 2022/0031729 A1
(43) Pub. Date: Feb. 3, 2022

(54) USE OF S-ADENOSYLMETHIONINE FOR PERSONALIZED TREATMENT OF DEPRESSION

(71) Applicant: Zentar LLC, Dover, DE (US)

(72) Inventor: Tarek E. Ammar, Dover, DE (US)

(21) Appl. No.: 17/389,793

(22) Filed: Jul. 30, 2021

Related U.S. Application Data
(60) Provisional application No. 63/059,707, filed on Jul. 31, 2020.

(51) Int. Cl. A61K 31/7076

(52) U.S. Cl. CPC A61K 31/7076

(57) ABSTRACT

The present invention relates to personalized treatment of depression using S-adenosylmethionine composition.

### FIELD OF THE INVENTION

The present invention relates to personalized treatment of depression using S-adenosyl-L-methionine (SAMe).

### BACKGROUND OF THE INVENTION

Major depressive disorder (MDD) is a leading cause of disease burden in the world, and widely viewed as having inadequate treatment options. MDD affects nearly twenty million people in the United States—approximately one out of two patients fail to achieve an adequate response with antidepressant treatment and two thirds of patients do not experience remission in a timely manner. Moreover, the WHO reports that less than 50 percent of people worldwide suffering with depression receive treatment for their condition. Current prescription treatments for depression are known to produce often significant and undesirable side effects. Further, currently approved augmentation therapy for inadequate response has undesirable tolerability and side effects. S-adenosyl-L-methionine (SAMe), an endogenous molecule produced in all body tissues, exhibits pharmacological activity in animal model models of depression, although its exact mechanism of action is unknown. As an endogenous molecule, SAMe exhibits a side effect profile markedly more tolerable than current prescription depression treatments.

There is some evidence that SAMe is effective in treating depression in humans, but results have been mixed and contradictory. Recently, and in the most significant trial to date, a SAMe formulation was tested in a multicenter, randomized, double-blind, placebo-controlled Phase II clinical trial to compare the efficacy and safety of the formulation in combination with ongoing antidepressant therapy against placebo plus ongoing antidepressant therapy for treating MDD. The SAMe formulation did not exhibit a significant treatment difference versus placebo. Accordingly, there is a need in the art for an effective way to identify and treat MDD patients using SAMe.

### SUMMARY OF THE INVENTION

Provided herein is a method of treating depression in a subject, which comprises administering a composition comprising S-adenosyl-L-methionine (SAMe) to the subject. Also provided are uses of a composition comprising SAMe for treating depression, or for the manufacture of a medicament for treating depression. The subject may have been identified (that is, may be characterized) as having one or more of the following biomarkers:

(a) a single nucleotide polymorphism (SNP) at position 677 of SEQ ID NO: 1 or position 27 of SEQ ID NO: 7 (identified by rs1801133) comprising at least one thymine (T) allele or the complement thereof, wherein the SEQ ID NO: 1 and SEQ ID NO: 7 are each independently a portion of a genomic nucleic acid sequence of methylenetetrahydrofolate reductase (MTHFR);

(b) a SNP at position 1298 of SEQ ID NO: 1 comprising at least one cytosine (C) allele or the complement thereof;

(c) a SNP at position 1793 of SEQ ID NO: 1 or position 27 of SEQ ID NO: 8 (identified by rs2274976) comprising at least one adenosine (A) allele or the complement thereof, wherein the SEQ ID NO: 1 and SEQ ID NO: 8 are each independently a portion of a genomic nucleic acid sequence of MTHFR;

(d) a SNP at position 2756 of SEQ ID NO: 2 or position 27 of SEQ ID NO: 9 (identified by rs1805087) comprising at least one guanosine (G) allele or the complement thereof, wherein the SEQ ID NO: 2 and SEQ ID NO: 9 are each independently a portion of a genomic nucleic acid sequence of methionine synthase (MTR);

(e) a SNP at position 66 of SEQ ID NO: 3 or position 27 of SEQ ID NO: 10 (identified by rs1801394) comprising at least one G allele or the complement thereof, wherein the SEQ ID NO: 3 and SEQ ID NO: 10 are each independently a portion of a genomic nucleic acid sequence of methionine synthase reductase (MTRR);

(f) a SNP at position 27 of SEQ ID NO: 11 (identified by rs1006737) comprising at least one A allele or the complement thereof, wherein the SEQ ID NO: 11 is a portion of a genomic nucleic acid sequence of calcium channel, voltage-dependent, L type, alpha 1C subunit (CACNA1C);

(g) a SNP at position 27 of SEQ ID NO: 12 (identified by rs1883729) comprising at least one A allele or the complement thereof, wherein the SEQ ID NO: 12 is a portion of a genomic nucleic acid sequence of DNA (cytosine-5)-methyltransferase 3 beta (DNMT3B);

(h) a SNP at position 27 of SEQ ID NO: 13 (identified by rs7163862) comprising at least one T allele or the complement thereof, wherein the SEQ ID NO: 13 is a portion of a genomic nucleic acid sequence of GTP cyclohydrolase 1 feedback regulatory protein (GCHFR);

(i) a SNP at position 27 of SEQ ID NO: 14 (identified by rs12659) comprising at least one T allele or the complement thereof, wherein the SEQ ID NO: 14 is a portion of a genomic nucleic acid sequence of reduced folate carrier protein (RCF2);

(j) a SNP at position 27 of SEQ ID NO: 15 (identified by rs202676) comprising at least one G allele or the complement thereof, wherein the SEQ ID NO: 15 is a portion of a genomic nucleic acid sequence of folate hydrolase (prostate-specific membrane antigen) 1 (FOLH1);

(k) a SNP at position 27 of SEQ ID NO: 16 (identified by rs2297291) comprising at least one A allele or the complement thereof, wherein the SEQ ID NO: 16 is a portion of a genomic nucleic acid sequence of reduced folate carrier protein (RCF1);

(l) a SNP at position 27 of SEQ ID NO: 17 (identified by rs1051266) comprising at least one A allele or the complement thereof, wherein the SEQ ID NO: 17 is a portion of a genomic nucleic acid sequence of reduced folate carrier protein (RCF1);

(m) a SNP at position 27 of SEQ ID NO: 18 (identified by rs8007267) comprising at least one T allele or the complement thereof, wherein the SEQ ID NO: 18 is a portion of a genomic nucleic acid sequence of GTP cyclohydrolase 1 (GCH1);

(n) a SNP at position 27 of SEQ ID NO: 19 (identified by rs7639752) comprising at least one A allele or the complement thereof, wherein the SEQ ID NO: 19 is a portion of a genomic nucleic acid sequence of choline-phosphate cytidylyltransferase A (PCYT1A);

(o) a SNP at position 27 of SEQ ID NO: 20 (identified by rs6275) comprising at least one T allele or the complement thereof, wherein the SEQ ID NO: 20 is a portion of a genomic nucleic acid sequence of dopamine receptor D2 (DRD2);

(p) a SNP at position 27 of SEQ ID NO: 21 (identified by rs1079596) comprising at least one T allele or the complement thereof, wherein the SEQ ID NO: 21 is a portion of a genomic nucleic acid sequence of dopamine receptor D2 (DRD2);

(q) a SNP at position 27 of SEQ ID NO: 22 (identified by rs11240594) comprising at least one A allele or the complement thereof, wherein the SEQ ID NO: 22 is a portion of a genomic nucleic acid sequence of dopamine receptor D2 (DRD2);

(r) a SNP at position 27 of SEQ ID NO: 23 (identified by rs4633) comprising at least one C allele or the complement thereof, wherein the SEQ ID NO: 23 is a portion of a genomic nucleic acid sequence of catechol-β-methyltransferase (COMT);

(s) a SNP at position 27 of SEQ ID NO: 24 (identified by rs4680) comprising at least one G allele or the complement thereof, wherein the SEQ ID NO: 24 is a portion of a genomic nucleic acid sequence of catechol-β-methyltransferase (COMT);

(t) a SNP at position 27 of SEQ ID NO: 25 (identified by rs250682) comprising at least one C allele or the complement thereof, wherein the SEQ ID NO: 25 is a portion of a genomic nucleic acid sequence of solute carrier family 6 (neurotransmitted transported, dopamine), member 3 (SLC6A3);

(u) a SNP at position 27 of SEQ ID NO: 26 (identified by rs2277820) comprising at least one T allele or the complement thereof, wherein the SEQ ID NO: 26 is a portion of a genomic nucleic acid sequence of formiminotransferase cyclodeaminase (FTCD);

(v) a SNP at position 27 of SEQ ID NO: 27 (identified by rs2236225) comprising at least one A allele or the complement thereof, wherein the SEQ ID NO: 27 is a portion of a genomic nucleic acid sequence of methylenetetrahydrofolate dehydrogenase (NADP+dependent) 1 (MTHFD1);

(w) an expression level ratio of S-adenosyl methionine (SAM) to S-adenosyl homocysteine (SAH) smaller than a pre-determined reference ratio;

(x) an expression level of 4-hydroxynonenal (4-HNE) greater than a first pre-determined reference value;

(y) an expression of high sensitive C-reactive protein (hsCRP) greater than a second pre-determined reference value;

(z) a homocysteine level greater than or equal to a third pre-determined reference value;

(aa) a folate level less than or equal to a fourth pre-determined reference value;

(bb) a B12 level less than a fifth pre-determined reference value;

(cc) a methylmalonic acid level greater than a sixth pre-determined reference value; and,

(dd) a formiminoglutamic acid level greater than or equal to a seventh pre-determined reference value.

The method may comprise the step of detecting the presence of the one or more biomarkers.

The SAMe may comprise a gallic acid ester, which may be methyl gallate, ethyl gallate, propyl gallate, butyl gallate, isobutyl gallate, isoamyl gallate, octyl gallate, dodecyl gallate, lauryl gallate, hexadecyl gallate, cetyl gallate, gallocatechol, gallocatechin. In particular, the gallic acid ester may be ethyl gallate, isoamyl gallate, propyl gallate, or octyl gallate, and even more specifically may be ethyl gallate, propyl gallate, or octyl gallate. The ratio (weight:weight) of gallic ester to SAMe ion may be from 5:1 to 1:400. The ratio may be from 1:1 to 1:100, or from 1:2 to 1:80. The composition may comprise from about 1 to about 400 mg of the gallic acid ester, about 5 to about 200 mg of the gallic acid ester, or from about 5 to about 100 mg of the gallic acid ester. The composition may comprise 0.1 to 40%, 0.1 to 25%, or 0.1 to 10% by weight of the gallic acid ester. The dose of SAMe ion may be from about 50 mg to about 3200 mg.

The composition may comprise an oral dosage form. The oral dosage form may comprise a functional coating, which may be an enteric coating, time-release coating, or pH-dependent coating. The composition may comprise a physiologically effective amount of SAMe. Non-parenteral administration of the composition to a selected human subject group may produce in the selected human subject group an average SAMe plasma area under the curve (average AUC) of at least 600 ng·h/mL per each 100 mg dosage of SAMe ion delivered and an average C max of at least 110 ng/mL per each 100 mg dosage of SAMe ion delivered. The composition may be manufactured at a relative humidity of less than 10%. The dosage from may contain less than or equal to 3.5% water, or less than or equal to 1.5% water.

The composition may comprise 60-80% SAMe, 0-10% disintegrant, 0-1% colloidal silicon dioxide, 0.1-2% lubricant, and at least 7% of a binder, wherein all % values are based on the percentage by weight of the composition. Oral administration of the composition to a selected human subject group may produce in the subject group: (a) an average maximum SAMe blood plasma concentration (average C max) of at least about 110 ng/mL per each 100 mg of SAMe ion in the composition; and (b) an average SAMe plasma area under curve (average AUC) within a range of about 400 ng·h/mL to about 800 ng·h/mL per each 100 mg of SAMe ion in the composition. The disintegrant may be may be sodium starch glycolate, croscarmellose sodium, crospovidone, or a combination thereof. The lubricant may be magnesium stearate, talc, calcium stearate, stearic acid, stearin hydrogenated vegetable oils, sodium benzoate, leucine, sodium stearyl fumarate, or a combination thereof. The binder may be a sugar, gelatin, a gum, microcrystalline cellulose, waxes, synthetic polymers, polyethylene glycol, polyvinyl pyrrolidone, or a combination thereof. The composition may comprise a tablet, paste, capsule, granule, caplet, or lozenge.

The composition when administered to the selected human subject group may provide in the subject group an average T max and/or C max with reduced variation. The composition may provide in the subject group an improved pharmacokinetic profile through: an equivalent average AUC to bi-daily dosing and/or reduced side effects through once a day dosing. Oral administration of the composition to the selected subject group may produce in the subject group an average C max of at least about 120 ng/mL per each 100 mg of SAMe ion in the composition.

The depression may be major depressive disorder. The subject may be resistant to treatment with a selective reuptake inhibitor (SSRI). The subject may also not be obese.

The subject may have been identified as having one or more of: (a) a SNP at position 27 of SEQ ID NO: 18 (rs8007267) comprising at least one T allele or the complement thereof; and, (b) a SNP at position 27 of SEQ ID NO. 24 (rs4680) comprising two guanine G alleles or the complement thereof. The subject may have been identified as having one or more of: (a) a SNP at position 1298 of SEQ ID NO: 1 comprising at least one C allele or the complement thereof; and, (b) a SNP at position 677 of SEQ ID NO: 1 or position 27 of SEQ ID NO: 27 comprising at least one T allele or the complement thereof.

### DETAILED DESCRIPTION

S-adenosyl-L-methionine (“SAM-e” or “SAMe”) is a naturally occurring compound that is present in almost every tissue throughout the body. Aside from water, SAMe is considered the second most common metabolic molecule, with adenosine triphosphate (ATP) being the most common. Supplementation with exogenous SAMe has been shown to be effective for treating of various ailments, including arthritis, Alzheimer's, and liver disease. But in clinical studies, SAMe has shown mixed results in its efficacy for treating depression.

The inventor has had the insight that selecting subjects for treatment with SAMe compositions based on certain biomarkers can lead to improved treatment efficacy against MDD. Subjects identified as having one or more single nucleotide polymorphisms and/or peripheral biomarker levels described herein, and/or who are not obese, may be more responsive to treatment with SAMe compositions, or responsive to lower doses of SAMe compositions.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Treatment” or “treating,” when referring to protection of a subject from a disease, means preventing, suppressing, repressing, or completely eliminating the disease. Preventing the disease involves administering a composition of the present invention to a subject prior to onset of the disease. Suppressing the disease involves administering a composition of the present invention to a subject after induction of the disease but before its clinical appearance. Repressing the disease involves administering a composition of the present invention to a subject after clinical appearance of the disease.

### 2. S-Adenosyl-L-Methionine Compositions

Provided herein are compositions comprising S-adenosyl-L-methionine or derivatives thereof. A SAMe composition referred to herein may comprise SAMe, one or more derivatives thereof, or a combination of the foregoing.

a. S-adenosylmethionine

As used herein the term “SAMe” refers to S-adenosyl-L-methionine (or, more simply, “S-adenosylmethionine”), a salt thereof, or a combination of the foregoing. The SAMe may have the following structure:

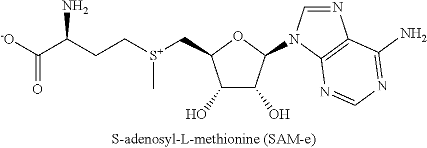

When referring to dose or percentage, the amount (typically in mg) refers to the dose of SAMe ion administered. SAMe is most commonly available as a stable salt form, e.g. with p-toluenesulfonic acid (see U.S. Pat. No. 3,893,999, incorporated herein by reference in its entirety). Other stable SAMe salts are described in, for example, U.S. Pat. No. 5,128,249, which describes particular stable salts of SAMe. Various forms of SAMe are suitable for use in certain embodiments provided herein. Thus, as used herein, “SAMe” refers to one or more of a stable salt, amorphous form, semicrystalline form, crystalline form, and ionic form of SAMe, or a combination thereof. The SAMe may be a form of SAMe that is known to be present in vivo. Amorphous forms of SAMe can be employed at any particle size and particle size distribution.

b. SAMe Derivatives

SAMe derivatives may comprise a modification, which may comprise an esterification. For example, SAMe derivative may comprise a gallic acid ester. The SAMe derivative may be as described in one or more of U.S. Pat. Nos. 8,975,238 and 9,925,208, the contents of which are incorporated herein by reference. The gallic acid ester may be methyl gallate, ethyl gallate, propyl gallate, butyl gallate, isobutyl gallate, isoamyl gallate, octyl gallate, dodecyl gallate, lauryl gallate, hexadecyl gallate, cetyl gallate, gallocatechol, gallocatechin, and epigallocatechin. In some embodiments, the gallic acid ester is ethyl gallate, isoamyl gallate, propyl gallate, or octyl gallate. In particular, the gallic acid ester may be ethyl gallate, isoamyl gallate, octyl gallate, or propyl gallate.

“Gallates,” “alkyl gallates” or “gallic acid esters” as used herein refer to salts and esters of gallic acid and have the general formula I (see below) where R₁ is a hydrocarbon chain which may be straight or branched. Optionally, R₁ group may be an alkyl, alkenyl, alkynyl, aryl, benzyl, phenyl, alicyclic, or heterocyclic group all of which groups may be substituted or unsubstituted. R₁ is preferably a C₁-C₂₂ alkyl group, a C₂-C₂₂ alkenyl group or a C₂-C₂₂ alkynyl group, all of which groups may be substituted or unsubstituted and may be straight chain, cyclic, cyclic unsaturated or branched chain. Moreover, this hydrocarbon chain can be a saturated, monounsaturated, or polyunsaturated. R₁ may be a saturated hydrocarbon chain ranging from C₁-C₂₂.

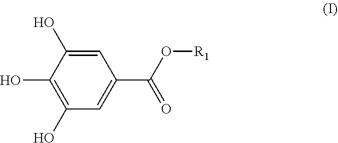

The gallic acid ester may comprise methyl gallate, ethyl gallate, propyl gallate, iso-propyl gallate, butyl gallate, isobutyl gallate, amyl gallate, isoamyl gallate, hexyl gallate, isohexyl gallate, heptyl gallate, isoheptyl gallate, octyl gallate, isooctyl gallate, nonyl gallate, isononyl gallate, decyl isodecyl, undecyl gallate, isoundecyl gallate, dodecyl gallate (lauryl gallate), isododecyl gallate, tridecyl gallate, isotridecyl, tetradecyl gallate, isotetradecyl gallate, pentadecyl gallate, isopentadecyl gallate, hexadecyl gallate (cetyl gallate), isohexadecyl gallate, heptadecyl gallate, isoheptadecyl gallate, octadecyl gallate, isoctadecyl gallate, cis-9-hexadecenyl (palmitoleyl) gallate, cis-9-octadecenyl (oleyl) gallate, cis,cis-9,12 octadecadienyl (linoleyl) gallate, trans,trans-9,12-octadecadienyl (linolelaidyl) gallate, cis,cis,cis-9,12,15-octadecatrienyl (linolenyl) gallate, trans,trans,trans-9,12,15-octadecatrienyl (linolenelaidyl) gallate, cis,cis,cis-6,9,12-octadecatrienyl (gamma-linolenyl) gallate, trans 9-octadecenyl (elaidyl) gallate or trans-9-hexadecenyl (palmitelaidyl) gallate. catechin gallate, epicatechin gallate, gallocatechin gallate, epigallocatechin gallate, gallocatechol gallate, 2-ethylhexyl gallate, 2-hydroxyethyl gallate, 6-O-galloylglucose, hamamelitannin, methoxyethoxyethoxyethyl m-digallate, theaflavin monogallate A&B, theaflavin digallate. The gallic acid ester may be ethyl gallate, isoamyl gallate, propyl gallate or octyl gallate. The gallic acid ester may be considered a GRAS (Generally Recognized As Safe) substance by the U.S. Food and Drug Administration (FDA). The gallic acid ester may have received a Novel Food approval by either the European Food Safety Authority (EFSA) or the European Medicines Agency (EMA). The gallic acid ester may be propyl gallate. Thus provided herein are formulations comprising SAMe and propyl gallate. In one example, the gallic acid ester is propyl gallate.

The ratio (weight:weight) of gallic acid ester to exogenous SAMe may be from 5:1 to 1:400. In some examples, the ratio (weight:weight) of gallic acid ester to S-adenosylmethionine is from 5:1 to 1:100, from 4:1 to 1:80, or from 1:1 to 1:16. The gallic acid ester may be ethyl gallate, isoamyl gallate or octyl gallate and the weight ratio of ethyl gallate, isoamyl gallate or octyl gallate to SAMe is from 1:1 to 1:16. In one example, the gallic acid ester is propyl gallate and the weight ratio of propyl gallate:SAMe is from 1:1 to 1:100. In another example, the gallic acid ester is propyl gallate and the weight ratio of propyl gallate:SAMe is from 1:1 to 1:16. In a further example, the gallic acid ester is propyl gallate and the weight ratio of propyl gallate:SAMe is from 1:1 to 1:2, 1:2 to 1:3, 1:3 to 1:4, 1:4 to 1:5, 1:5 to 1:6, 1:6 to 1:7, 1:7 to 1:8, 1:8 to 1:9, 1:9 to 1:10, 1:10 to 1:11, 1:11 to 1:12, 1:12 to 1:13, 1:13 to 1:14, 1:14 to 1:15, or 1:15 to 1:16. The gallic acid ester may be propyl gallate and the weight ratio of propyl gallate:SAMe may be about 1:16. In one example, the SAMe composition comprises SAMe and propyl gallate wherein the weight ratio of propyl gallate:SAMe is from 1:1 to 1:16. The composition may comprise SAMe and propyl gallate, wherein the weight ratio of propyl gallate:SAMe is about 1:16.

The composition may comprise from about 1 to about 5 mg, or about 5 to about 10 mg of gallic acid ester. The composition may also comprise about 10-50 mg gallic acid ester. The composition may comprise from about 50 to about 100 mg, about 100 to about 150 mg, about 150 to about 200 mg, about 200 to about 250 mg, about 250 to about 300 mg, about 300 to about 350 mg, or about 350 to about 400 mg gallic acid ester. The composition may comprise from about 5-100 mg of ethyl gallate, isoamyl gallate, propyl gallate or octyl gallate. The composition may comprise from about 5-100 mg of propyl gallate. The SAMe composition may be administered such that the daily amount of propyl gallate dosed does not exceed the acceptable daily intake (“ADI”) for propyl gallate as established by the Joint FAO/WHO Expert Committee on Food Additives (JECFA).

The SAMe composition may comprise from 0.25 to 1%, 1 to 2%, 2 to 3%, 3 to 4%, 4 to 5%, 5 to 6% or 6 to 7% by weight gallic acid ester wherein the weight percentage is based on the weight of the total dosage form. The composition may comprise 7 to 10%, 10 to 15%, 15 to 20%, 20 to 25%, 25 to 30%, 30 to 35%, 35 to 40%, 40 to 50%, 50 to 60%, 60 to 70%, 70 to 80%, or greater than 80% by weight gallic acid ester.

The SAMe composition may be a “low-dose” SAMe composition. By increasing the uptake of exogenous SAMe in the presence of a gallic acid ester, the daily administered effective dose of SAMe may be substantially lowered by administration of compositions with improved SAMe uptake in comparison to control formulations that do not contain at least one gallic acid ester. These “low-dose” treatments may enable a lower daily pill count, while still achieving the same or better pharmacokinetics in comparison to previously available SAMe products administered on a bi-daily or greater schedule. Some embodiments relate to administration of a selected improved dosage on a once-a-day basis. In some embodiments, the once-a-day dose may be administered in a single dosage unit exemplified by, a single tablet, capsule, or caplet. The single dose may be administered as multiple tablets, capsules or caplets taken at one time. A dosage of about 400 to 3200 mg of SAMe ion per day may be divided into two, three, four or more tablets, capsules or caplets of about 50 to 2000 mg, preferably about 100 to 1600 mg of SAMe per unit. The daily dose may comprise two, three or four units (e.g. tablets, capsules or caplets) of about 100 to 800 mg of SAMe ion per unit. Suitable dosage regimens included are: four units of about 50-400 mg of SAMe ion per unit, e.g. 50, 100, 150, 200, 250, 300, 350 or 400 mg SAMe ion per unit; three units of about 50-1000 mg of SAMe ion per unit, e.g. 50, 100, 150, 200, 250, 300, 350, 400, 450, 500, 550, 600, 650, 700, 750, 800, 850, 900, 950 or 1,000 mg of SAMe ion per unit; two units of about 50-3200 mg of SAMe ion per unit, e.g. about 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, or 3200 mg of SAMe ion per unit.

SAMe exemplary formulations comprising a gallic acid ester may be configured to enable high bioavailability of the exogenous SAMe. “High bioavailability” formulations are those which provide higher average maximum SAMe blood plasma concentration (C max) and/or average SAMe plasma area under the curve (AUC) values in comparison to the same dosage forms of SAMe without the gallic acid ester or in comparison to other currently available commercial SAMe formulations. High bioavailability formulations when dosed to a selected subject group provide an average C max of at least about 100 to 130 ng/mL (and/or an average AUC of at least about 500 ng·h/mL) per each 100 mg dosage of SAMe ion. The SAMe composition may be provided in a high bioavailability SAMe formulation.

The composition when administered to a selected subject group may provide in the subject group an average SAMe C max (average maximum plasma concentration) of at least about 100 ng/mL per each 100 mg of SAMe ion, at least about 110 ng/mL per each 100 mg of SAMe ion, or at least about 120 ng/mL per each 100 mg of SAMe ion, or of at least about 130 ng/mL per each 100 mg of SAMe ion, or of at least about 150 ng/mL per each 100 mg of SAMe ion, or of at least about 175 ng/mL per each 100 mg of SAMe ion, or of at least about 200 ng/mL per each 100 mg of SAMe ion, or of at least about 225 ng/mL per each 100 mg of SAMe ion or of at least about 250 ng/mL per each 100 mg of SAMe ion, or of at least about 300 ng/mL per each 100 mg of SAMe ion. The composition when administered to the selected subject group may provide in the subject group an average SAMe C max of at least about 12 ng/mL, at least about 13 ng/mL, at least about 15 ng/mL, at least about 17.5 ng/mL, at least about 20 ng/mL, at least about 22.5 ng/mL, at least about 25 ng/mL, or at least about 30 ng/mL per each 10 mg of SAMe ion. The SAMe composition when administered to the selected subject group may provide in the subject group an average SAMe C max of at least about 1.2 ng/mL, at least about 1.3 ng/mL, at least about 1.35 ng/mL, at least about 1.5 ng/mL, at least about 1.75 ng/mL, at least about 2.0 ng/mL, at least about 2.25 ng/mL, at least about 2.5 ng/mL, or at least about 3.0 ng/mL per each 1 mg of SAMe ion. The gallic acid ester may be propyl gallate. The composition when administered to the selected subject group may provide in the subject group an average SAMe C max of at least about 1.2 ng/mL, at least about 1.3 ng/mL, at least about 1.35 ng/mL, at least about 1.5 ng/mL, at least about 1.75 ng/mL, at least about 2.0 ng/mL, at least about 2.25 ng/mL, at least about 2.5 ng/mL, or at least about 3.0 ng/mL per each 1 mg of SAMe ion.

The SAMe composition when administered to selected subject group may provide in the subject group an average AUC of at least about 800 ng·h/mL per each 100 mg dosage of SAMe ion, or of at least about 850 ng·h/mL per each 100 mg dosage of SAMe ion, or at least about 900 ng·h/mL per each 100 mg dosage of SAMe ion, at least about 950 ng·h/mL per each 100 mg dosage of SAMe ion, or at least about 1000 ng·h/mL per each 100 mg dosage of SAMe ion. The composition when administered to the selected subject group may provide in the subject group an average SAMe AUC of at least about 80 ng·h/mL, at least about 85 ng·h/mL, at least about 90 ng·h/mL, at least about 95 ng·h/mL, or at least about 100 ng·h/mL per each 10 mg of SAMe ion. The composition may also provide an average SAMe AUC of at least about 8 ng·h/mL, at least about 8.5 ng·h/mL, at least about 9 ng·h/mL, at least about 9.5 ng·h/mL, or at least about 10 ng·h/mL per each 1 mg of SAMe ion. In one example, the gallic acid ester is propyl gallate. The composition may also provide an average SAMe AUC of at least about 8 ng·h/mL, at least about 8.5 ng·h/mL, at least about 9 ng·h/mL, at least about 9.5 ng·h/mL, or at least about 10 ng·h/mL per each 1 mg of SAMe ion. In another example, the dose of SAMe ion delivered is at least 10 mg. The dose of SAMe ion delivered may be from 10 to 1600 mg.

c. Formulation

The SAMe composition may have an “improved pharmacokinetic profile” or “enhanced pharmacokinetic profile” as used herein refers to one or more of the following criteria in comparison to conventional oral SAMe treatments: 1) high average C max (greater than about 1800 ng/mL when tested at a dose of 1600 mg (SAMe ion)); and/or 2) increased AUC (greater than about 7500 ng·h/mL when tested at a dose of 1600 mg (SAMe ion) or greater than about 4000 ng·h/mL when tested at a dose of 800 mg); and/or 3) pharmacokinetic parameters with reduced variation; and/or 4) reduced effective dose (for example, a C max of at least about 100 ng/mL per 100 mg dose of SAMe ion and/or an AUC of about 450 ng·h/mL per 100 mg dose of SAMe ion). The SAMe composition may provide a blood plasma SAMe C max of greater than about 2000 ng/mL, greater than about 3000 ng/mL, or greater than about 3500 ng/mL when tested at a dose of 1600 mg (SAMe ion). The SAMe composition may provide a C max of at least about 110 ng/mL per 100 mg dose of SAMe ion, at least about 130 ng/mL per 100 mg dose of SAMe ion, at least about 150 ng/mL per 100 mg dose of SAMe ion, at least about 180 ng/mL per 100 mg dose of SAMe ion, at least about 210 ng/mL per 100 mg dose of SAMe ion, or at least about 240 ng/mL per 100 mg of SAMe ion. The SAMe composition may provide an AUC of greater than about 8000 ng·h/mL, greater than about 10000 ng·h/mL, greater than about 11000 ng·h/mL, or greater than about 12000 ng·h/mL when tested at a dose of 1600 mg (SAMe ion). The composition may provide an AUC of at least 500 ng·h/mL per 100 mg SAMe dosed, at least 600 ng·h/mL per 100 mg SAMe dosed, at least 700 ng·h/mL per 100 mg SAMe dosed, or at least 800 ng·h/mL per 100 mg SAMe dosed.

The SAMe composition may be as described in one or more of U.S. Pat. Nos. 8,329,208; 8,865,203; 8,580,296; and, 9,931,356, the contents of which are incorporated herein by reference.

The SAMe composition may be suitable for non-parental administration, which may be oral. Formulations for non-parenteral administration of drugs/therapeutic agents are typically provided as solid or semi-solid products or dosage forms, exemplified by tablets, capsules or pellets, and generally consist of a core “matrix material” which ‘encapsulates’ the drug as well as one or more protective coatings. “Product” or “dosage form” as used herein refers to any solid or semi-solid formulation or preparation used for non-parental administration. Non-parenteral formulations or preparations as described herein include oral delivery systems exemplified by tablets, pastes, capsules, granules, caplets, lozenges and the like; and transmucosal or inhaled delivery systems, exemplified by aerosols, irrigants, topical creams, pastes, lozenges, patches, and the like, all of which are well-known and well-documented in the art. These formulations may be administered using a clinical, pharmaceutical or veterinary dosing regimen. Non-parenteral dosage forms may also be provided as medical foods or dietary or nutritional supplements. Non-parenterally administered SAMe formulations may be configured to enable extended release of the formulated SAMe.

Upon administration, the rate of release of an active moiety from a non-parenteral product can be greatly influenced by the excipients and/or product characteristics which make up the product itself. For example, an enteric coat on a tablet is designed to separate that tablet's contents from the stomach contents to prevent, for example, degradation of the stomach which may induce gastrointestinal discomfort or injury. SAMe and other tablets described in the art are commonly enteric coated. Once the dosage form has transited from the stomach to the duodenum and subsequently the rest of the small intestine the enteric coat is removed due the pH change and the table disintegrates/dissolves according to its intrinsic properties and the dosage form technologies that have been applied. According to the currently accepted conventional understanding, systemic exposure of the active moiety will be relatively insensitive to the small formulation changes. For example, following the conventional understanding, one would expect similar or near similar PK behavior for formulations with an application of enteric coat within the normal range of recommended application thicknesses. Similarly, following the conventional understanding, one would expect comparable behavior for dosage forms prepared within a range of humidity that allowed for the efficient and effective handling of the active moiety through the course of the formulation process. For SAMe as the active moiety, the impact of these processing parameters is expected to be particularly blunted in light of the teaching within the art that SAMe is subject to low bioavailability due to extensive first pass metabolism. Thus one would predict consistent systemic exposure as measured by pharmacokinetics for different enteric coated dosage forms within the normal range of operating parameters as long as active moiety was delivered intact to the site of absorption in the small intestine. Combinations of exogenous SAMe with one or more excipients and/or processing parameters which result in specific product characteristics can dramatically affect the pharmacokinetic profile of SAMe and lead to high C max and AUC values in vivo.

Suitable excipients which result in improved pharmacokinetic profiles of SAMe are preferably included in non-parenteral formulations of the invention. More specifically, formulations which include SAMe and one or more suitable excipients, exemplified by matrix materials, binders, lubricants, glidants or disintegrants which aid in modulating the PK profile of administered exogenous SAMe are preferred. Other embodiments of the invention relate to compositions comprising SAMe in combination with one or more suitable excipients and one or more specific product characteristics (such as dissolution or water content) which result in improved pharmacokinetic profiles of SAMe in vivo. Thus, the in vivo performance of non-parenteral SAMe dosage forms/products included herein is based upon the composition of the excipients added during manufacturing and/or the final product characteristics generated through specific processing parameters and methods.

Product or Dosage Form Characteristics

The product or dosage form characteristics which result from the processing methods and/or parameters for generating non-parenteral formulations such as tablets, include, but are not limited to, hardness, thickness, water content, friability, disintegration, dissolution profile(s), shape, size, weight, uniformity and composition. These product characteristics can be modulated in a number of ways and affect the final in vitro and/or in vivo performance of the formulations. As an example, tablets generated by compression or molding processes may have varying degrees of thickness or hardness depending on the processing parameters under which they were made. Product or dosage form characteristics may be a consequence of excipient selection, excipient composition, manufacturing methods applied or a combination of any of these. The combination of excipients as well as product characteristics (including processing methods or processing parameters) of the final dosage form will ultimately determine the pharmacokinetic profile of the active ingredient in vivo. SAMe formulations may be processed or manufactured under specific conditions such as, for example, mixing methods (including sieve size, rpm, and milling), drying time, press conditions, environmental parameters (e.g. temperature and humidity) and combinations thereof) which themselves modulate the pharmacokinetic profile of SAMe in vivo (i.e. increase the average C max or AUC). In order to quantitatively compare one tablet to another, it is customary to measure several of these product or dosage form characteristics. This is also necessary when attempting to duplicate multiple batches.

A specific “window” of dissolution (i.e. a particular amount of drug release over a certain time frame) may be correlated with those formulations of the invention which exhibited an improved SAMe PK profile in vivo. Although dissolution studies are commonly utilized to characterize non-parenteral formulations, testing is standard using a buffer phase which is at pH 6.8 to best represent the pH of the distal small intestine.

Dissolution and drug release from formulations depends on many factors including the solubility and concentration of the active ingredient, the nature and composition of the excipients, content uniformity, water content, product shape and size, porosity, disintegration time and other factors. The release of a drug or active ingredient from a final dosage form in vitro is typically characterized by its dissolution profile under standardized conditions (using United States Pharmacopeia (USP) or similar accepted methods for reference) and at pH 6.8 as mentioned above. The dissolution profile shows the amount of drug released over time into the test media under specified conditions. Standard conditions make use of buffers at pH 6.8 in order to best mimic the pH of distal small intestine. The dissolution test method for enteric dosage forms involves incubation of the formulation in a first acidic phase for two hours and is then transferred to the aqueous buffer phase (pH 6.8). Time points for measuring drug release begin at this two-hour time period (i.e. when first transferred into the aqueous buffer phase). When dissolution profiles of multiple SAMe formulations are analyzed under conditions of pH 6.8, all formulations may show “fast” dissolution and their dissolution profiles may not be distinguishable from one formulation to the next. When dissolution studies are conducted using a pH 6.0 buffer, which best mimics that the pH of a specific region of the upper small intestine where SAMe is absorbed, there may be a significant differentiation between formulations that are “fast” to dissolve and release drugs. Moreover, pharmacokinetic analysis of these formulations in vivo may show that formulations of the invention dissolving rapidly within a specific “window,” as seen in the pH 6.0 dissolution profiles, may correlate with those formulations exhibiting very high C max and AUC values

An improved pharmacokinetic SAMe composition may show targeted dissolution in a buffer phase of pH 6.0. Between 25-80% SAMe may be released after one hour of incubation in the buffer phase; about 30-70% SAMe may be released within one hour of incubation in the buffer phase; or, about 30-60% SAMe is released within one hour of incubation in the buffer phase.

Excipients are usually grouped by their function such as: disintegrants, diluents, binders, lubricants, glidants, coatings, coloring agents or flavoring agents, and the same excipient may be used for more than one function in a given oral formulation. Commonly used pharmaceutically acceptable excipients include water, magnesium stearate, starch, lactose, microcrystalline cellulose, stearic acid, sucrose, talc, silicon dioxide, gelatin, acacia and dibasic calcium phosphate (Baldrick, P. (2000) Regul. Toxicol. Pharmacol. October 32(2):210). Excipients are combined with active ingredients for example to enhance appearance, improve stability, aid processing or aid disintegration after administration, but many other excipient functions are known in the art that can be applied to SAMe oral dosage forms. Classes of excipients which are often used and suitable for use in the present invention include but are not limited to, natural, modified-natural or synthetic mono-, oligo- or polysaccharides where oligo- and polysaccharides may or may not be physically or chemically crosslinked; natural, modified-natural or synthetic mono-, oligo- and polypeptides or proteins where oligo- and polypeptides and proteins may or may not be physically or chemically crosslinked; synthetic oligomers and polymers that may or may not be physically or chemically crosslinked; monomeric, hydrophobic, hydrophilic or amphoteric organic molecules; inorganic salts or metals; and combinations thereof. Accordingly, SAMe may be combined with any excipient(s) known in the art that allows tailoring its performance during manufacturing as well as its in vitro and in vivo performance. Many of these excipients may be utilized to tailor the dissolution profiles of SAMe formulations.

Disintegrants may be added to non-parenteral formulations to induce breakup of the product or dosage form (i.e. tablet or capsule) when it comes in contact with aqueous fluid in order to help release the drug. The objectives behind addition of disintegrants are to increase surface area of the product fragments and to overcome cohesive forces that keep these particles together in a formulation. They do this by promoting wetting and swelling of the dosage form so that it breaks up in the gastrointestinal tract. Some binders such as starch and cellulose also act as disintegrants. Other disintegrants are clays, cellulose derivatives, algins, gums and crosslinked polymers. Another group of disintegrants called “super-disintegrants” are often utilized. These materials are effective at low (2-5%) concentrations. “Super-disintegrants” which may be suitable for use in the present invention include, but are not limited to, sodium starch glycolate (SSG), croscarmellose sodium or crosprovidone.

The SAMe composition may comprise SAMe and one or more disintegrants or “super-disintegrants” which improve the pharmacokinetic profile of SAMe in vivo.

The binding material which holds the bulk of the product together and also helps maintain the product in a desired shape is known as a “binder” or “granulator.” Binders suitable for use in the present invention are exemplified by, but are not limited to, sugars, gelatin, gums, microcrystalline cellulose and modified celluloses, waxes or synthetic polymers like polyethylene glycol or polyvinyl pyrrolidone.

The SAMe composition may comprise SAMe and one or more binders.

Additional excipients often utilized in product formulations are lubricants. These are substances which aid in the manufacturing process as they help minimize clumping of the products and also help release them from the manufacturing machinery. The most common “lubricant” used for oral formulations is magnesium stearate; however, other commonly used product lubricants include talc, calcium stearate, stearic acid (stearin), hydrogenated vegetable oils, sodium benzoate, leucine, carbowax 4000 and sodium stearyl fumarate all of which may be suitable for use in the present invention.

The SAMe composition may comprise SAMe and one or more lubricants.

Glidants also referred to as “flow-aids,” help to keep the powder making up the products flowing as the products are being made, stopping them from forming lumps. Examples of commonly used glidants which may be suitable for use in the invention include colloidal silicon dioxide, talc, calcium silicate and magnesium silicate.

The SAMe composition may comprise SAMe and one or more glidants.

An outer coating is typically applied to oral dosage forms in order to mask taste, odor or color; provide physical or chemical protection for the active ingredient/drug; control the release of the active ingredient from the formulation; protect the active ingredient from the harsh environment of the stomach (i.e. enteric coating); or protect the subject from unwanted gastrointestinal side effects. Prior to applying the external coating, a seal coating may first be applied. Seal coatings act to smooth the product surfaces, enhance the adherence of the final, outer coat and/or to protect the active ingredient from premature degradation. The type and/or thickness of the seal coat or the final coating(s) may be varied in order to alter product characteristics, such as dissolution. Typically, the external or functional coatings are targeted to be about 4-10% by weight and seal coats are targeted to be about 1-5%, preferably about 2%, by weight. Seal coats are generally thought of as “non-functional” in that they are not utilized to control timing or placement of release of the active ingredient; however, it is considered that certain seal coatings may act as such “functional” coatings. For the purpose of the present invention, “functional coatings” are intended to include enteric coatings, time-release coatings, pH-dependent coatings or other which control the timing or placement of release of the active ingredient. In some exemplified embodiments of the invention, the one or more separate coatings or layers of the functional coating together constitute 5% or less of the total dosage form targeted by weight. In preferred embodiments the functional coating is an enteric coating and even more preferably the enteric coating is about 3-4% targeted by weight.

The SAMe composition may comprise SAMe and one or more coatings which alter the pharmacokinetic parameters of exogenous SAMe. Other embodiments of the invention may include improved pharmacokinetic compositions comprising SAMe and one or more coatings which alter the in vitro dissolution profile of SAMe. Specific exemplified embodiments of the invention include improved pharmacokinetic compositions comprising SAMe and one or more coatings which result in a dissolution profile exhibiting about 25-80% SAMe released after one hour of incubation in the buffer phase; particularly about 30-70% SAMe released within one hour of incubation in the buffer phase; and even more particularly, about 30-60% SAMe released within one hour of incubation in the buffer phase.

The suitability of a particular excipient, such as, for example, a “matrix material,” “disintegrant,” “super-disintegrant” “binder,” “lubricant,” “glidant,” or “coating” may be identified by analyzing the in vivo pharmacokinetics of formulations containing the excipient and SAMe. Alternatively, in vitro analysis of one or more excipients using a series of standard in vitro techniques which are well known in the art may be used to pre-screen excipients and ultimately provide a means to predict in vivo pharmacokinetic profiles. Furthermore, the use of references in the art may also provide insight into potentially suitable pharmaceutically or nutritionally acceptable excipients (such as a “matrix material,” “disintegrant,” “binder,” “lubricant,” “glidant,” or “coating”) for use in the present invention. In vitro analysis of one or more excipients using dissolution studies conducted with a buffer pH of less than 6.8 may be used to pre-screen excipients and ultimately provide a means to predict in vivo pharmacokinetic profiles.

d. Processing Methods and Parameters

Processing methods and/or parameters which may be modified in order to improve the pharmacokinetic profile and/or alter the dissolution profile of SAMe compositions include but are not limited to: relative humidity, temperature, drying time and other environmental parameters.

The SAMe composition may be generated under low humidity conditions, typically less than or equal to about 35%, less than or equal to about 15-25%, or less than or equal to about 10%. The SAMe composition may also generated under manufacturing conditions wherein the temperature is maintained between about 15-35° C. The SAMe composition may be manufactured using a drying time of about 4-24 hours. The SAMe composition may comprise low water content. “Low water content” includes those formulations containing less than or equal to about 5% water, less than or equal to about 3.5% water, or less than or equal to about 1.5% water. In one way, water content is altered by controlling the relative humidity during the manufacturing process.

The SAMe composition may comprise exogenous SAMe and one or more suitable excipients and/or processing parameters which improve the pharmacokinetic profile of SAMe in vivo. The improved pharmacokinetic profile may be identified by an average C max of SAMe of at least about 1800 ng/mL, at least about 1900 ng/mL or at least about 2000 ng/mL, when tested at a 1600 mg dose of SAMe ion; an AUC of at least about 7500 ng·h/mL, 8000 ng·h/mL, 8500 ng·h/mL, or 9000 ng·h/mL when tested at a 1600 mg dose of SAMe ion; a T max or C max with reduced variation; a reduced effective dose, or combinations thereof. The improved pharmacokinetic profile may be an average C max of SAMe of at least 1800 ng/mL for a 1600 mg dose of SAMe ion. The improved pharmacokinetic profile may be identified by an average C max of SAMe of at least about 800 ng/mL, 825 ng/mL, 850 ng/mL, 875 ng/mL, 900 ng/mL, at least about 950 ng/mL or at least about 1000 ng/mL, when tested at a 800 mg dose of SAMe ion; an AUC of at least about 3000 ng·h/mL, 3250 ng·h/mL, 3500 ng·h/mL, or 3750 ng·h/mL when tested at a 800 mg dose of SAMe ion; a T max or C max with reduced variation; a reduced effective dose, or combinations thereof. The improved pharmacokinetic profile may be identified by an average C max of SAMe of at least about 400 ng/mL, 425 ng/mL, 450 ng/mL, at least about 475 ng/mL or at least about 500 ng/mL, when tested at a 400 mg dose of SAMe ion; an AUC of at least about 1500 ng·h/mL, 1550 ng·h/mL, 1600 ng·h/mL, or 1650 ng·h/mL when tested at a 400 mg dose of SAMe ion; a T max or C max with reduced variation; a reduced effective dose, or combinations thereof. The improved pharmacokinetic profile may be identified by an average C max of SAMe of at least about 400 ng/mL, at least about 450 ng/mL or at least about 500 ng/mL, when tested at a 400 mg dose of SAMe ion; an AUC of at least about 1500 ng·h/mL, 1550 ng·h/mL, 1600 ng·h/mL, or 1650 ng·h/mL when tested at a 400 mg dose of SAMe ion; a T max or C max with reduced variation; a reduced effective dose, or combinations thereof. The improved pharmacokinetic profile may also be identified by an average C max of SAMe of at least about 100 ng/mL per 100 mg dose of SAMe ion. Similarly, the improved pharmacokinetic profile may also be identified by an AUC of about 450 ng·h/mL per 100 mg dose of SAMe ion.

e. Administration

The SAMe composition may be used for a treatment described herein in combination with an antidepressant. The antidepressant may be administered in the same composition as the SAMe. The antidepressant drug and the SAMe may be administered in separate compositions at the same time (e.g., concurrently) or sequentially (e.g., one after the other), or in any temporal administration regimen, where the SAMe increases the efficacy of the antidepressant drug as compared to the efficacy without the SAMe. The anti-depressant drug and/or the SAMe may be administered in a single dose or in divided doses. The number of dosages administered over a period of time (e.g., per day) for the antidepressant drug and the SAMe may be the same or different. The antidepressant drug and the SAMe may be administered via the same or different routes.

The anti-depressant drug may be a selective serotonin reuptake inhibitor (SSRI). The SSRI may comprise one or more of fluoxetine, citalopram, paroxetine, escitalopram, and sertraline.

### 2. Methods of Treatment

Provided herein is a method of treating depression in a human subject with a SAMe composition described herein. The depression may be major depressive disorder. The treatment may comprise administering the SAMe composition to the subject. The subject may have been determined to exhibit one or more characteristics described herein, which may include one or more biomarkers and/or obesity. A treatment described herein may also comprise determining the presence of one or more of the characteristics, and administering the SAMe composition to the subject. The treatment may also comprise administering the composition to a subject exhibiting one or more of the characteristics.

The treatment efficacy of the SAMe composition for the subject (e.g., the responsiveness of the subject to one or more of the SAMe and SAMe derivatives) may be predicted by determining the presence of one or more biomarkers of the subject. The biomarkers may be as described in U.S. Pat. No. 9,546,401, the contents of which are incorporated herein by reference. A biomarker or combination thereof may indicate that the subject is more responsive to treatment with the SAMe composition, or may be responsive to treatment with a reduced dosage of the SAMe composition as compared to a subject who does not exhibit the biomarker(s). A biomarker may be one or more single nucleotide polymorphisms (SNPs) in a gene (that is, may be a genotypic biomarker). A biomarker may also comprise levels of a protein, metabolite, or chemical, or a ratio thereof, in a sample obtained from the subject. Additionally, obesity may indicate that the subject may be less responsive to SAMe treatment. Accordingly, if the subject is not obese, then the subject may be more responsive to SAMe.

a. Genotypic Biomarkers

The SNP may be identified by a Reference SNP cluster ID (rs) number as follows: rs1801133 present in methylenetetrahydrofolate reductase (MTHFR); rs2274976 present in MTHFR; rs1805087 present in methionine synthase (MTR); rs1801394 present in methionine synthase reductase (MTRR); rs1006737 present in calcium channel, voltage-dependent, L-type, alpha 1C subunit (CACNA1C); rs1883729 present in DNA (cytosine-5)-methyltransferase 3 beta (DNMT3B); rs7163862 present in GTP cyclohydrolase 1 feedback regulatory protein (GCHFR); rs12659 present in reduced folate carrier protein (RCF2); rs202676 present in folate hydrolase (prostate-specific membrane antigen) (FOLH1); rs2297291 present in reduced folate carrier protein (RCF1); rs1051266 present in reduced folate carrier protein 1 (RCF1); rs8007267 present in GTP cyclohydrolase 1 (GCH1); rs7639752 present in choline-phosphate cytidylytransferase A (PCYT1A); rs6275 present in dopamine receptor D2 (DRD2); rs1079596 present in DRD2; rs11240594 present in DRD2; rs4633 present in catechol-β-methyltransferase (COMT); rs4680 present in COMT; rs250682 present in dopamine active transporter (DAT, or SLC6A3); rs2277820 present in formiminotransferase cyclodeaminase (FTCD); rs2236225 present in methylenetetrahydrofolate dehydrogenase (NADP+dependent) 1 (MTHFD1); and any combinations thereof.

The genotype predictive of the subject's responsiveness to the SAMe treatment may comprise one or more of the following:

(i) a single nucleotide polymorphism (SNP) at position 677 of SEQ ID NO: 1 or position 27 of SEQ ID NO: 7 (identified by rs1801133) comprising at least one T allele or the complement thereof, wherein the SEQ ID NO: 1 and SEQ ID NO: 7 are each independently a portion of a genomic nucleic acid sequence of methylenetetrahydrofolate reductase (MTHFR);

(ii) a SNP at position 1298 of SEQ ID NO: 1 comprising at least one C allele or the complement thereof;

(iii) a SNP at position 1793 of SEQ ID NO: 1 or position 27 of SEQ ID NO: 8 (identified by rs2274976) comprising at least one A allele or the complement thereof, wherein the SEQ ID NO: 1 and SEQ ID NO: 8 are each independently a portion of a genomic nucleic acid sequence of MTHFR;

(iv) a SNP at position 2756 of SEQ ID NO: 2 or position 27 of SEQ ID NO: 9 (identified by rs1805087) comprising at least one G allele or the complement thereof, wherein the SEQ ID NO: 2 and SEQ ID NO: 9 are each independently a portion of a genomic nucleic acid sequence of methionine synthase (MTR);

(v) a SNP at position 66 of SEQ ID NO: 3 or position 27 of SEQ ID NO: 10 (identified by rs1801394) comprising at least one G allele or the complement thereof, wherein the SEQ ID NO: 3 and SEQ ID NO: 10 are each independently a portion of a genomic nucleic acid sequence of methionine synthase reductase (MTRR);

(vi) a SNP at position 27 of SEQ ID NO: 11 (identified by rs1006737) comprising at least one A allele or the complement thereof, wherein the SEQ ID NO: 11 is a portion of a genomic nucleic acid sequence of calcium channel, voltage-dependent, L type, alpha 1C subunit (CACNA1C);

(vii) a SNP at position 27 of SEQ ID NO: 12 (identified by rs1883729) comprising at least one A allele or the complement thereof, wherein the SEQ ID NO: 12 is a portion of a genomic nucleic acid sequence of DNA (cytosine-5)-methyltransferase 3 beta (DNMT3B);

(viii) a SNP at position 27 of SEQ ID NO: 13 (identified by rs7163862) comprising at least one T allele or the complement thereof, wherein the SEQ ID NO: 13 is a portion of a genomic nucleic acid sequence of GTP cyclohydrolase 1 feedback regulatory protein (GCHFR);

(ix) a SNP at position 27 of SEQ ID NO: 14 (identified by rs12659) comprising at least one T allele or the complement thereof, wherein the SEQ ID NO: 14 is a portion of a genomic nucleic acid sequence of reduced folate carrier protein (RCF2);

(x) a SNP at position 27 of SEQ ID NO: 15 (identified by rs202676) comprising at least one G allele or the complement thereof, wherein the SEQ ID NO: 15 is a portion of a genomic nucleic acid sequence of folate hydrolase (prostate-specific membrane antigen) 1 (FOLH1);

(xi) a SNP at position 27 of SEQ ID NO: 16 (identified by rs2297291) comprising at least one A allele or the complement thereof, wherein the SEQ ID NO: 16 is a portion of a genomic nucleic acid sequence of reduced folate carrier protein (RCF1);

(xii) a SNP at position 27 of SEQ ID NO: 17 (identified by rs1051266) comprising at least one A allele or the complement thereof, wherein the SEQ ID NO: 17 is a portion of a genomic nucleic acid sequence of reduced folate carrier protein (RCF1);

(xiii) a SNP at position 27 of SEQ ID NO: 18 (identified by rs8007267) comprising at least one T allele or the complement thereof, wherein the SEQ ID NO: 18 is a portion of a genomic nucleic acid sequence of GTP cyclohydrolase 1 (GCH1);

(xiv) a SNP at position 27 of SEQ ID NO: 19 (identified by rs7639752) comprising at least one A allele or the complement thereof, wherein the SEQ ID NO: 19 is a portion of a genomic nucleic acid sequence of choline-phosphate cytidylyltransferase A (PCYT1A);

(xv) a SNP at position 27 of SEQ ID NO: 20 (identified by rs6275) comprising at least one T allele or the complement thereof, wherein the SEQ ID NO: 20 is a portion of a genomic nucleic acid sequence of dopamine receptor D2 (DRD2);

(xvi) a SNP at position 27 of SEQ ID NO: 21 (identified by rs1079596) comprising at least one T allele or the complement thereof, wherein the SEQ ID NO: 21 is a portion of a genomic nucleic acid sequence of dopamine receptor D2 (DRD2);

(xvii) a SNP at position 27 of SEQ ID NO: 22 (identified by rs11240594) comprising at least one A allele or the complement thereof, wherein the SEQ ID NO: 22 is a portion of a genomic nucleic acid sequence of dopamine receptor D2 (DRD2);

(xviii) a SNP at position 27 of SEQ ID NO: 23 (identified by rs4633) comprising at least one C allele or the complement thereof, wherein the SEQ ID NO: 23 is a portion of a genomic nucleic acid sequence of catechol-β-methyltransferase (COMT);

(xix) a SNP at position 27 of SEQ ID NO: 24 (identified by rs4680) comprising at least one G allele or the complement thereof, wherein the SEQ ID NO: 24 is a portion of a genomic nucleic acid sequence of catechol-β-methyltransferase (COMT);

(xx) a SNP at position 27 of SEQ ID NO: 25 (identified by rs250682) comprising at least one C allele or the complement thereof, wherein the SEQ ID NO: 25 is a portion of a genomic nucleic acid sequence of solute carrier family 6 (neurotransmitted transported, dopamine), member 3 (SLC6A3);

(xxi) a SNP at position 27 of SEQ ID NO: 26 (identified by rs2277820) comprising at least one T allele or the complement thereof, wherein the SEQ ID NO: 26 is a portion of a genomic nucleic acid sequence of formiminotransferase cyclodeaminase (FTCD);

(xxii) a SNP at position 27 of SEQ ID NO: 27 (identified by rs2236225) comprising at least one A allele or the complement thereof, wherein the SEQ ID NO: 27 is a portion of a genomic nucleic acid sequence of methylenetetrahydrofolate dehydrogenase (NADP+dependent) 1 (MTHFD1));

The SNPs described herein can include one or two SAMe composition-responsive alleles. By way of example only, a SNP at position 677 of SEQ ID NO: 1 or position 27 of SEQ ID NO: 7 (identified by rs1801133) can comprise one T allele, or two T alleles. Without wishing to be bound by theory, a human subject determined to carry two SAMe-responsive alleles in a SNP locus described herein may show greater response to a SAMe composition than a subject with one SAMe-responsive allele in the same SNP locus.

Depending on the design of primers and probes, the SNPs described herein can be also represented by alleles complementary to the corresponding SAMe-responsive alleles described herein. For example, instead of detecting the presence of a SNP at position 677 of SEQ ID NO: 1 or position 27 of SEQ ID NO: 7 (identified by rs1801133) comprising at least one T allele, one of skill in the art can readily design primers and/or probes for the complementary sequence of SEQ ID NO: 7 to probe for a SNP at the same location comprising at least one A allele instead. Accordingly, the presence of at least one characteristic (including, e.g., at least two characteristics, at least three characteristics, at least four characteristics or more) described herein can be indicated by detecting the presence of the complementary allele of the SAMe-responsive allele

The subject may exhibit one or two SAMe composition-responsive alleles. In one example, the subject's genome may comprise a T allele at one or both of the SNP at position 677 of SEQ ID NO: 1 and position 27 of SEQ ID NO: 7 (identified by rs1801133).

In particular, the subject's genome may comprise at least one T allele at position 677 of SEQ ID NO: 1 (or position 27 of SEQ ID NO: 7); and at least one G allele present at position 2756 of SEQ ID NO: 2 (or position 27 of SEQ ID NO: 9). In another example, the subject's genome comprises (i) a SNP at position 27 of SEQ ID NO: 18 (rs8007267) comprising at least one T allele or the complement thereof; and, (ii) a SNP at position 27 of SEQ ID NO: 24 (rs4680) comprising two G alleles or the complement thereof.

In another example, the subject has at least one T allele present at position 677 of SEQ ID NO: 1 (or position 27 of SEQ ID NO: 7); and, at least one G allele present at position 2756 of SEQ ID NO: 2 (or position 27 of SEQ ID NO: 9).

The subject may have a SNP at position 1298 of SEQ ID NO: 1 comprising at least one C allele, wherein the presence of the SNP at position 1298 of the SEQ ID NO: 1 comprising at least one C is indicative of the subject being more responsive to treatment with a SAMe composition.

In one example, the subject has a SNP at position 1298 of SEQ ID NO: 1 comprising at least one C allele, and a SNP at position 677 of SEQ ID NO: 1 or position 27 of SEQ ID NO: 27 comprising at least one T allele, which may indicate that the subject is more responsive to treatment with a SAMe composition.

In particular, a subject positive for the MTR 2756 AG or GG genotype [associated single-nucleotide polymorphism (SNP) rs1805087] may be more responsive to the SAMe composition compared to subjects negative for this SNP. A subject positive for the COMT CC (rs4633) or GG (rs4680) SNP may also be more responsive to the SAMe composition. Thus, the presence of the MTR (and COMT) SNP biomarkers may allow identification of subjects who are responsive to the SAMe composition, when comparing treated subjects vs. placebo as well as when comparing biomarker positive and biomarker negative subjects within the treatment.

In one example, the subject has a SNP at position 2756 of SEQ ID NO: 2 or position 27 of SEQ ID NO: 9 (identified by rs1805087) comprising at least one B allele or the complement thereof, wherein the SEQ ID NO: 2 and SEQ ID NO: 9 are each independently a portion of a genomic nucleic acid sequence of methionine synthase (MTR), based on the recognition that the presence of the SNP allele(s) is associated with positive-symptom-reducing response to the SAMe composition. The subject may further have a SNP at position 677 of SEQ ID NO: 1 or position 27 of SEQ ID NO: 7 (identified by rs1801133) comprising at least one T allele or the complement thereof, wherein the SEQ ID NO: 1 and SEQ ID NO: 7 are each independently a portion of a genomic nucleic acid sequence of methylenetetrahydrofolate reductase (MTHFR).

In another example, the subject has a SNP at position 27 of SEQ ID NO: 23 (identified by rs4633) comprising two C alleles or the complement thereof, wherein the SEQ ID NO: 23 is a portion of a genomic nucleic acid sequence of catechol-O-methyltransferase (COMT), based on the recognition that the presence of the SNP allele(s) is associated with positive-symptom-reducing response to the SAMe composition.

The subject may have a SNP at position 27 of SEQ ID NO: 24 (identified by rs4680) comprising two G alleles or the complement thereof, wherein the SEQ ID NO: 24 is a portion of a genomic nucleic acid sequence of catechol-O-methyltransferase (COMT), based on the recognition that the presence of the SNP allele(s) is associated with positive-symptom-reducing response to the SAMe composition.

In another example, the subject has a SNP at position 27 of SEQ ID NO: 18 (rs8007267) comprising at least one thymine T allele or the complement thereof, wherein the SEQ ID NO: 18 is a portion of a genomic nucleic acid sequence of GTP cyclohydrolase 1 (GCH1); and a SNP at position 27 of SEQ ID NO: 24 (rs4680) comprising two guanine G alleles or the complement thereof, wherein the SEQ ID NO: 24 is a portion of a genomic nucleic acid sequence of catechol-O-methyltransferase (COMT).

In a further example, the subject has a SNP at position 677 of SEQ ID NO: 1 or position 27 of SEQ ID NO: 7 as identified as rs1801133 comprising at least one T allele for MTFHR; a SNP at position 2756 of SEQ ID NO: 2 or position 27 of SEQ ID NO: 9 as identified as rs1805087 comprising at least one guanine G allele for MTR; a SNP at position 27 of SEQ ID NO: 18 as identified as rs8007267 comprising at least one T allele for GCH1; and, a SNP at position 27 of SEQ ID NO: 24 as identified as rs4680 comprising two G alleles for COMT.

The subject's genotype may be determined by amplifying a test sample from the subject with a set of primers flanking any one of the SNPs described herein. At least two (e.g., at least three, at least four, at least five or more) sets of primers amplifying at least two (e.g., at least three, at least four, at least five or more) of the SNPs can be used in a multiplex amplification assay. A genotyping assay can comprise the step of amplifying the test sample with a set of primers flanking any one of the SNPs described herein. In some embodiments, at least two (e.g., at least three, at least four, at least five or more) sets of primers amplifying at least two (e.g., at least three, at least four, at least five or more) of the SNPs can be used in a multiplex amplification assay.

The gene sequences described herein are listed in the table below.

TABLE 1

| SEQ | | | Chromo- | |
|---|---|---|---|---|
| ID | | | some | Gene |
| NO | Sequence | rs number | locus | name |
| 7 | CTTGAAGGAGAAGGT | rs1801133 | 1p36.3 | MTHFR |
| | GTCTGCGGGAG[C/T] | | | |
| | CGATTTCATCATCACG | | | |
| | CAGCTTTTC | | | |
| 8 | CGAGGCCTTTGCCCT | rs2275976 | 1p36.3 | MTHFR |
| | GTGGATTGAGC[A/G] | | | |
| | GTGGGGAAAGCTGTAT | | | |
| | GAGGAGGAG | | | |
| 9 | GGAAGAATATGAAGA | rs1805097 | 1q43 | MTR |
| | TATTAGACAGG[A/G] | | | |
| | CCATTATGAGTCTCTC | | | |
| | AAGGTAAGT | | | |
| 10 | CAGGCAAAGGCCATC | rs1801394 | 5p15.11 | MTRR |
| | GCAGAAGAAAT[A/G] | | | |
| | TGTGAGCAAGCTGTGG | | | |
| | TACATGGAT | | | |
| 11 | TAAGTTCCATTCCAT | rs1006737 | 12p13.33 | CACNA1C |
| | CTCAGCCCGAA[A/G] | | | (ca ion) |
| | TGTTTTCAGAGCCGGA | | | |
| | GACCTACACA | | | |
| 12 | CTGCTGCTGGTATCA | rs1883729 | 20q11.2 | DNMT38 |
| | GCCTGGAGGAA[A/G] | | | |
| | TGAGTGACATCAGTTC | | | |
| | TCAGCATTA | | | |
| 13 | AACCAATCACAACAA | rs7163862 | 15q15.1 | GCHFR |
| | GGCAGATAAAG[A/T] | | | (BH4) |
| | AGGATGAGTTGTCAGA | | | |
| | TTTGATAA | | | |
| 14 | GCTTCGGAGCTGGAG | rs12659 | 21q22.3 | RCF2 |
| | CGCATGAATCC[C/T] | | | |
| | GGCCCAGGCGGGAAGC | | | |
| | TGGGACACG | | | |
| 15 | AAGCTGAGAACATCA | rS202676 | 11p11.2 | FOLH1 |
| | AGAAGTTCTTA[C/T] | | | (GCPI1) |
| | AGTAAGTACATCCTCG | | | |
| | AAAGTTTAT | | | |
| 16 | GGGAGGGCACCCGCA | rs2297291 | 21q22.3 | RCP1 |
| | GAGGCCTGCGC[A/G] | | | |
| | CTGACACTGCTGAGTG | | | |
| | GCTCTGCTC | | | |
| 17 | TGACCCCGAGCTCCG | rs1051266 | 21q22.3 | RCP1 |
| | GTCCTGGCGGC[A/G] | | | |
| | CCTCGTGTGCTACCTT | | | |
| | TGCTTCTAC | | | |
| 18 | CAATAGGAGCGTGTG | rs8007267 | 14qWW.1- | GCH1 |
| | TTTGAACAGTA[C/T] | | q22.2 | (GH4) |
| | ACGCCAAACTTCAGTC | | | |
| | ATTCAAGTA | | | |
| 19 | GGCCTAATCAATCCT | rs7639752 | 3q29 | PCYT1A |
| | TCTCATCTTTT[A/G] | | | |
| | TACCCACCTTTTGCAG | | | |
| | GAAACCTGT | | | |
| 20 | CTGACTCTCCCCGAC | rS6275 | 11q23.2 | DRD2 |
| | CCGTCCCACCA[C/T] | | | |
| | GGTCTCCACAGCACTC | | | |
| | CCGACAGCC | | | |
| 21 | GTCCCTGCAGTTTAA | rs1079596 | 11q23.2 | DRD2 |
| | TTATCCTCAAC[A/G] | | | |
| | TTACTGCCATACCCTA | | | |
| | CATTTTTGG | | | |
| 22 | CTCACAGTTTGTGGT | rs11240594 | | DRD2 |
| | TGAGACTAAGT[A/G] | | | |
| | TGACAACAGTGGCACT | | | |
| | TTGTGGTCC | | | |
| 23 | ACCAAGGAGCAGCGC | rs4633 | 22q11.21- | COMT |
| | ATCCTGAACCA[C/T] | | q11.23\| | |
| | GTGCTGCAGCATGCGG | | 22q11.21 | |
| | AGCCCGGGA | | | |
| 24 | CCCAGCGGATGGTGG | rs4680 | 22q11.21- | COMT |
| | ATTTCGCTGGC[A/G] | | q11.23\| | |
| | TGAAGGACAAGGTGTG | | 22q11.21 | |
| | CATGCCTGA | | | |
| 25 | TAATATGGCCACCCC | rs250682 | 5p15.3 | SLC6A3 |
| | AACTTTCGTAT[C/G] | | | |
| | ATTACTGTTTGTGTGG | | | |
| | TATTATCTT | | | |
| 26 | ATCAGCCCTAGATGC | rs2277820 | 21q22.3 | FTCD |
| | TTGACCAGCTC[C/T] | | | |
| | TCGGGCCTCACCTCCT | | | |
| | GGTTCTTCC | | | |
| 27 | CTGGGCCAACAAGCT | rs2236225 | 14q24 | MTHPD1 |
| | TGAGTGCGATC[C/T] | | | |
| | GGTCTGCAATGATGGA | | | |
| | GGAATTGCC | | | |

Methylenetetrahydrofolate Reductase (MTHFR)

Methylenetetrahydrofolate reductase (MTHFR) is an enzyme that in humans is encoded by the MTHFR gene. SEQ ID NO: 1 corresponds to a portion of the genomic nucleic acid sequence of human wild-type or normal MTHFR gene obtained from NCBI database (NCBI Reference Sequence: NM_005957.4), wherein the nucleotide at position 677 and 1298 of SEQ ID NO: 1 are normal (e.g., wild-type) “C” allele and “A” allele, respectively. Methylenetetrahydrofolate reductase catalyzes the conversion of 5,10-methylenetetrahydrofolate to 5-methyltetrahydrofolate, a cosubstrate for homocysteine remethylation to methionine. Genetic variation in this gene has been previously shown to influence susceptibility to occlusive vascular disease, neural tube defects, colon cancer, acute leukemia, Alzheimer's or vascular dementia, and mutations in this gene are associated with methylenetetrahydrofolate reductase deficiency.

The mutation of the MTHFR nucleotide at position 677 of SEQ ID NO: 1 from “C” allele to “T” allele (C677T) results in a change of amino acid residue from alanine to valine at position 222 of the corresponding amino acid sequence (SEQ ID NO: 4). Such amino acid substitution encodes a thermolabile enzyme with reduced activity. People with the thermolabile form of such enzyme generally have increased levels of homocysteine in their blood. Accordingly, in some embodiments, an increase in levels of homocysteine in a blood sample of a subject can be indicative of a SNP at position 677 (e.g., C677T) of the MTHFR gene (or SEQ ID NO: 1). In some embodiments, detection of valine at position 222 (e.g., A222V) of the corresponding amino acid sequence (SEQ ID NO: 4), e.g., by mass spectrometry, can indicate a SNP at position 677 (e.g., C677T) of the MTHFR gene (or SEQ ID NO: 1).

At nucleotide 1298 of the MTHFR, there are generally two possibilities: “A” or “C”. MTHFR 1298A (leading to a Glu at amino acid residue 429) is the most common while 1298C (leading to an Ala substitution at amino acid 429) is less common. In some embodiments, detection of alanine at position 429 (E429A) of the corresponding amino acid sequence (SEQ ID NO: 4) can indicate a SNP at position 1298 of the MTHFR gene (or SEQ ID NO: 1). Without wishing to be bound by theory, previous studies on human recombinant MTHFR have reported that the protein encoded by 1298C cannot be distinguished from 1298A in terms of activity, thermolability, FAD release, or the protective effect of 5-methyl-THF. It is believed that the C mutation (e.g., A1298C) does not appear to affect the MTHFR protein or result in thermolabile MTHFR, or affect homocysteine levels.

Methods for detecting the SNPs of the MTHFR gene, e.g., C677T, A1298C, or G1793A are well known in the art, for examples, including the methods and primers used in U.S. Pat. No. 6,833,243, which is incorporated herein by reference.

Methionine Synthase (MTR)

Methionine synthase also known as MS, MeSe, MetH is an enzyme that in humans is encoded by the MTR gene (5-methyltetrahydrofolate-homocysteine methyltransferase). SEQ ID NO: 2 corresponds to a portion of the genomic nucleic acid sequence of human wild-type or normal MTR gene obtained from NCBI database (NCBI Reference Sequence: NM 000254.2), wherein the nucleotide at position 2756 of SEQ ID NO: 2 is normal (e.g., wild-type) “A” allele. This enzyme is responsible for the regeneration of methionine from homocysteine. Methionine synthase forms part of the S-adenosylmethionine (SAM) biosynthesis and regeneration cycle. A polymorphism in the MTR gene, an A-to-G transition at position 2756 (e.g., A2756G) of SEQ ID NO: 2 causes an amino acid substitution from aspartic acid to glycine at codon 919 (D919G) of the corresponding amino acid sequence (SEQ ID NO: 5). Accordingly, in some embodiments, a glycine at position 919 (e.g., D919G) of the corresponding amino acid sequence (SEQ ID NO: 5), e.g., by mass spectrometry, can indicate a SNP at position 2756 (e.g., A2756G) of the MTR gene (or SEQ ID NO: 2).

Methionine Synthase Reductase (MTRR)

Methionine synthase reductase, also known as MSR, is an enzyme that in humans is encoded by the MTRR gene. SEQ ID NO: 3 corresponds to a portion of the genomic nucleic acid sequence of human wild-type or normal MTRR gene obtained from NCBI database (NCBI Reference Sequence: NM_002454.2), wherein the nucleotide at position 66 of SEQ ID NO: 3 is normal (e.g., wild-type) “A” allele. Methionine is an essential amino acid required for protein synthesis and one-carbon metabolism. Its synthesis is catalyzed by the enzyme methionine synthase. Methionine synthase eventually becomes inactive due to the oxidation of its cob(I)alamin cofactor. Methionine synthase reductase regenerates a functional methionine synthase via reductive methylation, and is a member of the ferredoxin-NADP(+) reductase (FNR) family of electron transferases. MTRR polymorphism, an adenine-to-guanine mutation at position 66 (e.g., A66G) of SEQ ID NO: 3 converts an isoleucine to a methionine amino acid (122M) at position 22 of the corresponding amino acid sequence (SEQ ID NO: 6). Accordingly, in some embodiments, detection of methionine at position 22 (e.g., 122M) of the corresponding amino acid sequence (SEQ ID NO: 6), e.g., by mass spectrometry, can indicate a SNP at position 66 (e.g., A66G) of the MTRR gene (or SEQ ID NO: 3).

Catechol-O-Methyltransferase (COMT)

Catechol-O-methyltransferase is an enzyme responsible for the breakdown of dopamine and norepinephrine, e.g., in the prefrontal cortex. Met/Met are more rapid metabolizers than Val/Val subjects in which are associated with cognitive dysfunction and disease pathology. In some embodiments, a hypometholated state has led to an overexpression of COMT and greater executive dysfunction. COMT polymorphism (identified by rs4680: SEQ ID NO: 24), an adenine-to-guanine mutation at position 27 of SEQ ID NO: 24 converts a valine (Val) to a methionine (Met) amino acid (Val158Met) at the corresponding position of the amino acid sequence. Accordingly, in some embodiments, detection of methionine at position 158 (e.g., V22M) of the corresponding amino acid sequence (SEQ ID NO: 28), e.g., by mass spectrometry, can indicate a SNP at position 27 of SEQ ID NO: 24.

Reduced Folate Carrier 1 &2 (RCF1 and RCF2)

Reduced folate carrier 1 and 2 (at SLC19A1) are receptors that transport 5-MTHF across various membranes including the choroid plexus and blood brain barrier.

Dopamine receptor D2 (DRD2) (at SLC26A9)

Taq1B and H313H are dopamine receptor polymorphisms that effect dopamine transmission, receptor density, and antipsychotic response.

DNA (cytosine-5)-methyltransferase 3 beta (DNMT3B)

DNA (cytosine-5)-methyltransferase 3 beta is s gene encoding a DNA methyltransferase which is believed to function in de novo methylation, rather than maintenance methylation.

Choline-Phosphate Cytidylytransferase A (PCYT1A)

Choline-phosphate Cytidylytransferase A (PCYT1A) is an enzyme that aids in the transformation of phosphatidylcholine to choline.

GTP Cyclohydrolase I (GCH1)

GTP cyclohydrolase I is part of the folate and biopterin biosynthesis pathways. It is responsible for the hydrolysis of guanosine triphosphate (GTP) to form 7,8-dihydroneopterin 3′-triphosphate. GTPCH is encoded by the gene GCH1 and is the rate-limiting enzyme in tetrahydrobiopterin (THB, BH4) biosynthesis. GCH1 is an essential cofactor in monamine synthesis and NO production.

Folate Hydrolase (Prostate-Specific Membrane Antigen) (FOLH1)

FOH1 is also known as glutamate carboxypeptidase II, which is an enzyme that in humans is encoded by the FOLH1 (folate hydrolase 1) gene. GCPII is a class II membrane glycoprotein. It catalyzes the hydrolysis of N-acetylaspartylglutamate (NAAG) to glutamate and N-acetylaspartate (NAA).

Dopamine Active Transporter (DAT)

The dopamine transporter (also dopamine active transporter, DAT, SLC6A3) is a membrane-spanning protein that pumps the neurotransmitter dopamine out of the synapse back into cytosol, from which other transporters sequester DA and NE into vesicles for later storage and release.

GTP Cyclohydrolase 1 Feedback Regulatory Protein (GCHFR)

GTP cyclohydrolase 1 feedback regulatory protein is an enzyme that in humans is encoded by the GCHFR gene. GTP cyclohydrolase 1 feedback regulatory protein binds to and mediates tetrahydrobiopterin inhibition of GTP cyclohydrolase 1 which aids in the production of de novo BH4 production.

Calcium Channel, Voltage-Dependent, L Type, Alpha 1C Subunit (CACNA1C)

Gene CACNA1C encodes an alpha-1 subunit of a voltage-dependent calcium channel Calcium channels mediate the influx of calcium ions into the cell upon membrane polarization.

Formiminotransferase Cyclodeaminase (FTCD)

Formiminotransferase cyclodeaminase is an enzyme that catalyzes the conversion of formiminoglutamate and tetrahydrofolate into formiminotetrahydrofolate and glutamate.

Methylenetetrahydrofolate Dehydrogenase (NADP+Dependent) 1 (MTHFD 1)

Methylenetetrahydrofolate Dehydrogenase (NADP+Dependent) 1 is a tri-allelic gene that encodes a protein that possesses three distinct enzymatic activities, methylenetetrahydrofolate dehydrogenase, methenyltetrahydrofolate cyclohydrolase and formate-tetrahydrofolate ligase. Each of these activities catalyzes one of three sequential reactions in the interconversion of 1-carbon derivatives of tetrahydrofolate, which are substrates for methionine, thymidylate, and de novo purine syntheses. A common single nucleotide polymorphism (SNP) at nucleotide 1958 of the MTHFD1 gene (or at position 27 of SEQ ID 27) causes a “G” to “A” transition, which results in an arginine to glutamate substitution at amino acid position 653 in the synthetase domain of the enzyme (See, e.g., Hol et al., (1998) “Molecular genetic analysis of the gene encoding the trifunctional enzyme MTHFD (methylenetetrahydrofolate-dehydrogenase, methenyltetrahydrofolate-cyclohydrolase, formyltetrahydrofolate synthetase) in patients with neural tube defects.” Clin Genet. 53: 119-25).

S-Adenosyl Methionine (SAM) and S-Adenosyl Homocysteine (SAH).

S-adenosyl methionine, commonly known as SAM, or SAM-e, or AdoMet, is a natural compound found in all living cells. It is one of the most used enzymatic substrates in biochemical reactions, second only to the universal energy storage and transfer molecule, adenosyl triphosphate (ATP).

S-Adenosyl methionine is a common cosubstrate involved in methyl group transfers. It is made from adenosine triphosphate (ATP) and methionine by methionine adenosyltransferase. Transmethylation, transsulfuration, and aminopropylation are the metabolic pathways that use SAM. SAH is formed by the demethylation of S-adenosyl-L-methionine (SAM). Further details about SAM and SAH, including immunoassays for determining SAM, SAH and/or ratios thereof are described in U.S. Pat. App. NO: US 2009/0263879, which is incorporated herein by reference.

4-Hydroxynonenal (4-HNE)

4-Hydroxynonenal, or 4-hydroxy-2-nonenal or 4-HNE or HNE, (C9H16O2), is an α,β-unsaturated hydroxyalkenal which is produced by lipid peroxidation in cells. 4-HNE is the primary α,β-unsaturated hydroxyalkenal formed in this process. It is found throughout animal tissue, and in higher quantities during oxidative stress due to the increase in the lipid peroxidation chain reaction, due to the increase in stress events. 4-HNE has been believed to play a key role in cell signal transduction, in a variety of pathways from cell cycle events to cellular adhesion. 4-HNE is also considered as possible causal agents of numerous diseases, such as chronic inflammation, neurodegenerative diseases, adult respiratory distress syndrome, atherogenesis, diabetes and different types of cancer.

Protein residues known to react with 4HNE via 1,4-addition are Cys, His, and Lys. Thus, in some embodiments, expression levels of 4-HNE can be determined by measuring expression levels of 4-HNE adducts, e.g., 4-HNE-His. Commercial ELISA kits for measuring 4-HNE adducts, e.g., OxiSelect™ HNE-His Adduct ELISA Kit are available, e.g., from CellBioLabs

b. Peripheral Biomarkers

Alternatively, or in combination with the SNP-based biomarkers described herein, the biomarker that is indicative of greater responsiveness to SAMe treatment may be one or more peripheral biomarkers, which may include relative expression levels between S-adenosyl methionine (SAM) and S-adenosyl homocysteine (SAH); expression of 4-hydroxynonenal (4-HNE); expression of high-sensitivity c-reactive protein (hsCRP); homocysteine levels; folate levels; B12 levels; methylmalonic acid levels; and, levels of formiminoglutamic acid (FIGLU). In addition, the subject may not be obese, which may be an indicator of better responsiveness to SAMe treatment. The genetic polymorphisms, peripheral biomarkers and clinical features have been assessed on a human cohort exhibiting depressive disorder and resistance to anti-depressant monotherapies (e.g., has treatment-resistant depression (TRD), in particular, selective serotonin reuptake inhibitor (SSRI)-resistant depression).

The peripheral biomarker may include one or more of an expression level ratio of S-adenosylmethionine (SAM) to S-adenosyl homocysteine (SAH) smaller than a pre-determined reference ratio; an expression level of 4-hydroxynonenal (4-HNE) greater than a first pre-determined reference value; and, an expression of high sensitivity C-reactive protein (hsCRP) greater than a second pre-determined reference value.

The ratio of SAM to SAH as measured in the subject may be smaller than the pre-determined reference ratio, e.g., smaller than a control ratio of SAM/SAH as measured in biological samples of normal healthy subjects. The control ratio of SAM to SAH as measured in a serum sample of normal healthy subjects may range from about 4 to about 12. The control ratio of SAM to SAH as measured in a serum sample of normal healthy subjects may be about 7. The expression ratio of SAM to SAH may be smaller than 3.0, or smaller than about 2.8, as measured in a plasma sample. The expression ratio of SAM to SAH may be smaller than 2.71, as measured in a plasma sample. Depending on the test sample source, e.g., a blood sample vs. a cerebrospinal fluid sample, the pre-determined reference ratio for a blood plasma sample can be different from that for, e.g., a cerebrospinal fluid sample.

The expression of 4-HNE in the subject may be greater than the first pre-determined reference value, e.g., greater than a control value of 4-HNE as measured in biological samples of normal healthy subjects. The control value of 4-HNE as measured in a serum sample of the normal healthy subjects may be about 0.24 μmol per liter of serum, or about 0.04 mg per liter of serum. The expression of 4-HNE in the subject may be greater than about 3 mg per liter, or greater than about 3.2 mg per liter of plasma or higher as measured in a plasma sample, The expression of 4-HNE may be at least 3.28 mg per liter of plasma as measured in a plasma sample, or higher. Depending on the test sample source, e.g., a blood sample vs. a cerebrospinal fluid sample, the pre-determined reference value for a plasma sample can be different from that for, e.g., a cerebrospinal fluid sample.

The hsCRP expression level in the subject may be greater than a second pre-determined reference value, e.g., greater than a control value of hsCRP as measured in biological samples of normal healthy subjects. In one embodiment, the control value of hsCRP as measured in a serum sample of normal healthy subjects can range from about 0.5 mg per liter of serum to about 4.5 mg per liter of serum. In some embodiments, if the expression of hsCRP is greater than about 2.3 mg per liter of plasma, as measured in a plasma sample, then the subject may be more responsive to SAMe treatment. Depending on the test sample source, e.g., a blood sample vs. a cerebrospinal fluid sample, the hsCRP expression a plasma sample can be different from that in, e.g., a cerebrospinal fluid sample.

The homocysteine level, which may be total homocysteine levels, in the subject may be ≥10.0 μmol/L as measured by standard laboratory procedures from a plasma sample. In some embodiments, if the total homocysteine levels in the subject are in this range, then the subject may be more responsive to SAMe treatment.

The folate level in the subject may be ≤7.1 ng/mL if the subject is 5-9 years of age; ≤8.0 ng/mL if the subject is 10-17 years old; and if the subject is ≥18 years old, then the folate level may be <5.4 ng/mL. In some embodiments, if the folate level in the subject is in one of these respective ranges, then the subject may be more responsive to SAMe treatment. The folate may be measured by standard laboratory procedures from a serum sample.

The B12 level may be <350 pg/mL if the subject is 5-9 years old; <360 pg/mL if the subject is 10-17 years old; or <300 pg/mL if the subject is older than 17 years of age. In some embodiments, if the B12 level is in one of these respective ranges, then the subject may be more responsive to SAMe treatment. The B12 may be measured by standard laboratory procedures from a serum sample.

The methylmalonic acid level may be >318 nmol/L, as measured by standard laboratory procedures from a serum sample. In some embodiments, if the methylmalonic acid level is in this range, then the subject may be more responsive to SAMe treatment.

The formiminoglutamic acid (FIGLU) level may be ≥1.2 μg/mg creatinine in urine, as measured by standard laboratory procedures from a urine sample. In some embodiments, if the FIGLU level is in this range, then the subject may be more responsive to SAMe treatment.

For any of the biomarker levels provided herein, it is understood that equivalent measures from alternative types of subject samples are also contemplated. For example, levels provided herein for a serum sample also include the equivalent levels for a urine sample.

Alternatively, or in addition to having one or more of the characteristics described herein, the subject may not be obese. If the subject is not obese, then the subject may be more responsive to treatment with a SAMe composition described herein, or may be responsive to treatment with a lower dose of the SAMe composition. Methods of determining obesity in a human subject are known in the art and can include, but are not limited to, body mass index (BMI) measurement, measurement of abdominal fat (e.g., by waist circumference or waist-hip ratio), measurement of body fat, skinfold thickness, underwater weighing (densitometry), air-displacement plethysmography, computerized tomography (CT) and magnetic resonance imaging (MRI), and dual energy X-ray absorptiometry (DEXA), and any combinations thereof. A BMI value of at least 30 kg/m² may be indicative of obesity.

One or more of the following subject characteristics may be predictive of the subject's responsiveness to SAMe treatment:

i. an expression ratio of SAM to SAH smaller than a pre-determined reference ratio;

ii. expression of 4-hydroxynonenal (4-HNE) greater than a first pre-determined reference value;

iii. a SNP at position 677 of SEQ ID NO: 1 (or at position 27 of SEQ ID NO: 7) comprising at least one T allele, wherein the SEQ ID NO: 1 and SEQ ID NO: 7 are each independently a portion of a genomic nucleic acid sequence of methylenetetrahydrofolate reductase (MTHFR);

iv. a SNP at position 2756 of SEQ ID NO: 2 (or at position 27 of SEQ ID NO: 9) comprising at least one G allele, wherein the SEQ ID NO: 2 and SEQ ID NO: 9 are each independently a portion of a genomic nucleic acid sequence of methionine synthase (MTR); and,

v. a SNP at position 66 of SEQ ID NO: 3 (or at position 27 of SEQ ID NO: 10) comprising at least one G allele, wherein the SEQ ID NO: 3 and SEQ ID NO: 10 are each independently a portion of a genomic nucleic acid sequence of methionine synthase reductase (MTRR).

The subject more responsive to SAMe treatment may have at least the SNPs located at positions 677 and 2756 of the MTHFR and MTR loci, respectively. In one example, the subject is not obese (e.g., the subject has a BMI less than 30 kg/m²), and has the SNPs located at the positions 2756 and 66 of the MTR and MTRR loci, respectively. In another example, the subject is not obese, and has the SNP located at the position 2756 of the MTR locus. In a further example, the subject has at least the SAM/SAH ratio smaller than the pre-determined reference ratio and the SNP located at the position 2756 of the MTR locus. In another example, the subject has at least the 4-HNE expression greater than the first pre-determined reference value and the SNPs located at the positions 2756 and 66 of the MTR and MTRR loci, respectively.

c. Subject

The subject may be diagnosed with depression. In particular the subject may have MDD. The subject may be resistant to an SSRI treatment. The subject may be diagnosed with depression using the Montgomery-Asberg Depression Rating Scale (MADRS) and Hamilton rating scale for depression (HamD₁₇) derived from a combined MADRS-HamD₂₈ rating instrument, the patient-rated Inventory of Depressive Symptomatology (IDS-SR₃₀), and the Clinical Global Impression of Severity (CGI-S) scale (Hamilton, 1960, Guy, 1976; Montgomery and Asberg, 1979; Rush et al., 2000; Sackheim et al., 2003). The subject may have one or more of a total HAM-D₁₇ score ≥16, individual HAM-D₁₇ mood item score ≥2, and a patient self-rated IDS-SR30 score >28. The subject may have a <20% score fluctuation on the total HAM-D₁₇ or IDS-SR₃₀. Other methods of diagnosing depression are known in the art. For example, the subject may be diagnosed using functional magnetic resonance imaging (fMRI).

In some embodiments, the subject has been diagnosed with or suspected of having or developing depression. Accordingly, in some embodiments, subjects that have been diagnosed or suspected of having or developing with depression are selected prior to subjecting them to the assays, methods and/or compositions described herein. In some embodiments, a subject selected for a treatment regimen comprising a SAMe composition is being treated for depression. In some embodiments, the subject is specifically administered a SAMe composition alone, or in combination to enhance (or as an adjuvant) the effect of the antidepressant drug, and not for another reason.

In some embodiments, the subject is specifically administered a SAMe composition for treatment of depression, and not for another reason. For example, a human subject diagnosed as having, or have a risk for, depression may take an effective amount of a SAMe composition (with or without an anti-depressant). In such instance, a human subject amenable to the assays, methods, and/or compositions described herein may be specifically identified as having depression before performing the assays and/or methods described herein and/or administering the compositions described herein.

The phrase “having a risk for depression” or “suspected of having or developing depression” or “suspected of having or developing major depressive disorder” refers to a subject that presents one or more symptoms indicative of a depression including major depressive disorder (e.g., unexplained insomnia, fatigue, irritability, etc.) or is being screened for depression including major depressive disorder (e.g., during a routine physical), for example, in accordance with the criteria listed in DSM-IV or ICD-10 as discussed below.

As used herein, the term “depression” generally refers to a mental state of depressed mood characterized by feelings of sadness, despair and discouragement. In some embodiments, depression is a clinical symptom, and can include, but not limited to, major depressive disorder (including single episode and recurrent), unipolar depression, treatment-refractory depression, resistant depression, anxious depression and dysthymia (also referred to as dysthymic disorder). Further, the term “depression” can encompass any major depressive disorder (MDD), dysthymic disorder, mood disorders due to medical conditions with depressive features, mood disorders due to medical conditions with major depressive-like episodes, substance-induced mood disorders with depressive features and depressive disorder not otherwise specific as defined by their diagnostic criteria, as listed in the American Psychiatric Association's Diagnostic and Statistical Manual of Mental Disorders, 4th Edition (DSM-IV) or any later edition thereof, or the World Health Organization's International Statistical Classification of Diseases and Related Health Problems (ICD-10). In one embodiment, depression is major depressive disorder.

The DSM-IV and ICD-10 provides a common language and standard criteria for the classification of mental disorders, and have been commonly used by a suitably trained general practitioner, or by a psychiatrist or psychologist for diagnosis of depression including major depressive disorders. Symptoms of depression can include, but are not limited to, problems concentrating, remembering, and/or making decisions, changes in eating and/or sleeping habits, a loss of interest in enjoyable activities, difficulty going to work or taking care of daily responsibilities, feelings of guilt and/or hopelessness, slowed thoughts and/or speech, and preoccupation with thoughts of death or suicide. One of skill in the art can determine the score or rating of depression based on DSM-IV or ICD-10.

Other scales or criteria for classification of mental disorders known in the art, e.g., Maier or HAMD-7 scale, or social functioning questionnaire (SFQ), visual analogue scale (VAS), and/or cognitive and physical function questionnaire (CPFQ) can also be used to determine the degree of depression.

In some embodiments, the subject may have been diagnosed with or suspected of having or developing major depressive disorder. A major depressive episode is characterized by the presence of a severely depressed mood that persists for at least two weeks. Episodes can be isolated or recurrent and can be categorized by a skilled practitioner as mild (few symptoms in excess of minimum criteria), moderate, or severe (marked impact on social or occupational functioning).

The subject may have been diagnosed with depression (e.g., major depressive disorder (MDD)) and may be resistant to antidepressant monotherapy, i.e., a treatment for depression with a single antidepressant only. The phrase “resistant to antidepressant monotherapy” is used herein in reference to a subject with depression being resistant to at least one antidepressant in one or more classes. This includes subjects with depression that are resistant to at least two, at least three, at least four or more antidepressants in one or more classes. In some embodiments, subjects amenable to assays, methods and compositions described herein are subjects that have been diagnosed with major depressive disorder (MDD) and are resistant to at least one serotonin reuptake inhibitors (SRI), including at least one, at least two, at least three, at least four or more SRIs. In some embodiments, subjects amenable to assays, methods and compositions described herein are subjects that have been diagnosed with major depressive disorder (MDD) and are resistant to at least one selective serotonin reuptake inhibitor (SSRI), including at least two, at least three, at least four or SSRIs.

In some embodiments, subjects who are resistant to antidepressant monotherapy do not show a clinically-relevant reduction (e.g., as evaluated by a physician or a psychologist) in at least one symptom of depression from which they are suffering, after they have been administered with the antidepressant monotherapy for at least about 3 weeks or more, or up to about 3 weeks. Examples of symptoms of depression include, but are not limited to, low or depressed mood, anhedonia, low energy levels, guilt, decreased work and interests, psychomotor retardation, agitation, psychic anxiety, somatic anxiety, general somatic symptoms, reduced cognition or any combinations thereof.

In some embodiments, subjects who are resistant to antidepressant monotherapy do not show a clinically relevant reduction in at least one symptom of depression (e.g., 1, 2, 3, or more symptoms), after they have been administered with the antidepressant monotherapy for at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks or more. In some embodiments, subjects are determined to be treatment resistant if they do not show a clinically relevant reduction in at least one symptom of depression (e.g., 1, 2, 3, or more symptoms), after they have been administered with the antidepressant monotherapy for at least or up to about 3 weeks, at least or up to about 4 weeks, at least or up to about 5 weeks, at least or up to about 6 weeks, at least or up to about 7 weeks, at least or up to about 8, at least or up to about 9 weeks, at least or up to about 10 weeks, at least or up to about 11 weeks, or at least or up to about 12 weeks. The clinically relevant reduction in symptoms of depression can be evaluated by a physician or a psychologist.

In some embodiments, the subject may have been diagnosed with treatment-resistant depression (TRD) or treatment-refractory depression. As used interchangeably herein, the term “treatment-resistant depression” or “treatment-refractory depression” refers to a kind of depression that does not respond or is resistant to at least two or more anti-depressant drugs, e.g., at least three or more, or at least four or more anti-depressant drugs. In some embodiments, a subject is diagnosed with a treatment-resistant depression if the subject does not show a clinically relevant reduction in at least one symptom of depression described herein, after he/she has been administered with at least two or more anti-depressant drugs (either individually or in combination) for at least about 3 weeks or more, e.g., at least about 4 weeks, at least about 5 weeks, at least about 6 weeks, at least about 7 weeks, at least about 8 weeks, at least about 9 weeks, at least about 10 weeks, at least about 11 weeks, at least about 12 weeks or more. In some embodiments, a subject is diagnosed with a treatment-resistant depression if the subject does not show a clinically relevant reduction in at least one symptom of depression described herein, after he/she has been administered with at least two or more anti-depressant drugs (either individually or in combination) for up to about 12 weeks, including, e.g., up to about 11 weeks, up to about 10 weeks, up to about 9 weeks, up to about 8 weeks, up to about 7 weeks, up to about 6 weeks, up to about 5 weeks, up to about 4 weeks, or up to about 3 weeks. In some embodiments, a subject is diagnosed with a treatment-resistant depression if the subject does not show a clinically relevant reduction in at least one symptom of depression described herein, after he/she has been administered with at least two or more anti-depressant drugs (either individually or in combination) for at least or up to about 6 weeks, at least or up to about 7 weeks, at least or up to about 8 weeks, at least or up to about 9 weeks, at least or up to about 10 weeks, at least or up to about 11 weeks, or at least or up to about 12 weeks. In some embodiments, the treatment-resistant depression is diagnosed if the subject does not experience clinically relevant improvement in the symptoms of depression after at least or up to about 12 weeks on an antidepressant medication.

In some aspects of all the embodiments described herein, the term “treatment-resistant depression” can also be defined as failing to achieve remission after two treatments or two antidepressants within 4-12 weeks of time.

In some embodiments, the subject may have been diagnosed with treatment-resistant depression (TRD) or treatment-refractory depression and are currently taking non-medicine treatment for TRD, e.g., but not limited to, electroconvulsive therapy, vagus nerve stimulation, transcranial magnetic stimulation, and/or “talk” therapy. These subjects can be recommended for, or administered with, a treatment regimen comprising a SAMe composition, alone or in combination with a non-medicine treatment for TRD as described herein. In some embodiments, these subjects can be recommended for, or administered with, a treatment regimen comprising a SAMe composition, in combination with at least one antidepressant drug, and optionally non-medicine treatment for TRD. In these embodiments, the antidepressant drug that is recommended for, or administered to, the subjects with TRD, in combination with a SAMe composition, can be an antidepressant drug to which the subjects have previously shown resistance, or an antidepressant drug that the subject has never tried.

In some embodiments, the subject may have been in remission from depression and is now diagnosed with a relapse or a predisposition to a relapse. In other embodiments, the subject may have been diagnosed with depression and is currently taking at least an antidepressant.

As used herein, the terms, “patient,” “individual,” and “subject” are used interchangeably herein. A subject can be male or female. In some embodiments, the subjects amenable to the assays, methods and/or compositions described herein are female subjects.

d. Detecting Biomarkers

(1) Test Sample and Collection and Preparation Thereof.

Methods of collecting test samples for at least one analysis performed in the assays and/or methods described herein are well known to those skilled in the art. In some embodiments, a test sample subjected to analysis performed in the assays and/or methods described herein is derived from a biological sample of a subject. The term “biological sample” as used herein denotes a sample taken or isolated from a biological organism, e.g., cell lysate, a homogenate of a tissue sample from a subject or a fluid sample from a subject. The term “biological sample” also includes untreated or pre-treated (or pre-processed) biological samples. In some embodiments, the biological sample is a biological fluid, including, but not limited to, blood (including whole blood, plasma, cord blood and serum), lactation products (e.g., milk), amniotic fluids, sputum, saliva, urine, semen, cerebrospinal fluid, bronchial aspirate, perspiration, mucus, liquefied feces, synovial fluid, lymphatic fluid, tears, tracheal aspirate, and fractions thereof. In other embodiments, the biological sample may include cell lysate and fractions thereof. For example, cells (such as red blood cells, platelets, white blood cells and any cells circulating in the biological fluid described herein) can be harvested and lysed to obtain a cell lysate. In some embodiments, a test sample or a biological sample is a blood sample. In some embodiments, a test sample or a biological sample comprises a plasma sample. In some embodiments, a test sample or a biological sample comprises a saliva sample. In some embodiments, a test sample or a biological sample comprises a buccal sample. In some embodiments, a test sample or a biological sample comprises a urine sample. In some embodiments, a test sample or a biological sample comprises a cerebrospinal fluid sample.

A “biological sample” may contain cells from subject, but the term can also refer to non-cellular biological material, such as non-cellular fractions of blood, saliva, or urine, that can be used to measure plasma/serum biomarker expression levels or determine SNPs. In some embodiments, the sample is from a resection, biopsy, or core needle biopsy. In addition, fine needle aspirate samples can be used. Samples can be either paraffin-embedded or frozen tissue.

The sample may be obtained by removing a sample of cells from a subject, In one example, the sample includes previously isolated cells (e.g. isolated by another person). The biological sample may be freshly collected or previously collected.

A test sample or a biological sample may be a nucleic acid product amplified after polymerase chain reaction (PCR). The nucleic acid product may be one or more of DNA, RNA and mRNA, and may be isolated from a particular biological sample using any of a number of procedures, which are well-known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russel, Cold Spring Harbor Laboratory Press, 2001.

(2) SNPs

A major database of human SNPs is maintained at NCBI as dbSNP, and it contains data for unique human SNPs consisting of 1.78×10⁸ submitted SNP (identified by an “ss” number) and 5.2×10⁷ reference SNP (identified by an “rs” number), as of Build History 135: human_9606 based on NCBI human genome build 37.3. The rs numbers are unique, do not change, and allow analysis of the particularly identified SNP in any genetic sample. Throughout the specification, the SNPs described herein can also be identified by an “rs” number. For example, the SNP at position 677 of SEQ ID NO: 1 can be identified by rs 1801133; the SNP at position 1298 of SEQ ID NO: 1 can be identified by rs 1801131; the SNP at position 2756 of SEQ ID NO: 2 can be identified by rs 1805087; The SNP at position 66 of SEQ ID NO: 2 can be identified by rs 1801394. With the “rs” numbers known for each SNP, one of skill in the art will be able to determine the position of a specific SNP within a respective chromosome.

While a SNP could conceivably have three or four alleles, nearly all SNPs have only two alleles. Analysis of the SNPs identified herein generally relies on the two alleles that are listed in connection with each SNP. For example, the SNPs at the MTHFR locus described herein are each indicated to have two alleles, “C” or “T” at the position 677 of SEQ ID NO: 1, and “A” or “C” at the position 1298 of SEQ ID NO: 1, wherein SEQ ID NO: 1 is a portion of a genomic nucleic acid sequence of MTHFR. The presence of at least one allele “T” at position 677 of SEQ ID NO: 1 and/or at least one allele “C” at position 1298 of SEQ ID NO: 1 indicates that a subject with depression is recommended for a treatment regimen comprising a SAMe compositions. The SNP at the MTR locus described herein is indicated to have two alleles, “A” or “G.” The presence of at least one allele “G” at position 2756 of SEQ ID NO: 2, wherein SEQ ID NO: 2 is a portion of a genomic nucleic acid sequence of methionine synthase (MTR), indicates the subject recommended for a treatment regimen comprising a SAMe composition. The SNP at the MTRR locus described herein is indicated to have two alleles, “A” or “G.” The presence of at least one allele “G” at position 66 of SEQ ID NO: 3, wherein SEQ ID NO: 3 is a portion of a genomic nucleic acid sequence of methionine synthase reductase (MTRR), indicates the subject may be recommended for a treatment regimen comprising a SAMe composition.

Those skilled in the art will readily recognize that nucleic acid molecules can be double-stranded molecules and that reference to a particular site on one strand refers, as well, to the corresponding site on a complementary strand. In defining a SNP position, SNP allele, or nucleotide sequence, reference to an adenine “A,” a thymine “T” (uridine “U”), a cytosine “C,” or a guanine “G” at a particular site on one strand of a nucleic acid molecule also defines the thymine “T” (uridine “U”), adenine “A,” guanine “G,” or cytosine “C” (respectively) at the corresponding site on a complementary strand of the nucleic acid molecule. Thus, reference can be made to either strand to refer to a particular SNP position, SNP allele, or nucleotide sequence. Probes and primers can be designed to hybridize to either strand, and SNP genotyping methods disclosed herein can generally target either strand.

Accordingly, analysis of the opposite strand is described herein as well. For the opposite-strand analysis, the SNPs at the MTHFR locus is allele “A” at position 677 or allele “G” at position 1298 of the complementary sequence of SEQ ID NO: 1, wherein SEQ ID NO: 1 is a portion of a genomic nucleic acid sequence of methylenetetrahydrofolate reductase (MTHFR); while the SNP at the MTR locus is allele “C” at position 2756 of the complementary sequence of SEQ ID NO: 2, wherein SEQ ID NO: 2 is a portion of a genomic nucleic acid sequence of methionine synthase (MTR); and the SNP at the MTRR locus is allele “C” at position 66 of the complementary sequence of SEQ ID NO: 3, wherein SEQ ID NO: 3 is a portion of a genomic nucleic acid sequence of methionine synthase reductase (MTRR).

Identification method of SNPs can be of either a positive-type (inclusion of an allele) or a negative-type (exclusion of an allele). Positive-type methods determine the identity of a nucleotide contained in a polymorphic site, whereas negative-type methods determine the identity of a nucleotide not present in a polymorphic site. Thus, a wild-type site can be identified either as wild-type or not mutant. For example, at a biallelic polymorphic site where the wild-type allele contains thymine and the mutant allele contains cytosine, a site can be positively determined to be either thymine or cytosine or negatively determined to be not thymine (and thus cytosine) or not cytosine (and thus thymine)

The methods described herein may comprise determining whether a subject is homozygous for a polymorphism, heterozygous for a polymorphism, or lacking the polymorphism altogether (i.e. homozygous wildtype). As an exemplary embodiment only, a method to detect the C>T variance at position 677 of SEQ ID NO: 1, and a method for determining the allele, heterozygous for the C- and T-alleles, or homozygous for the C-allele or the T-allele at the SNP loci are provided. Substantially any method of detecting any allele of the SNPs described herein, such as restriction enzyme digestion, allele-specific probe hybridization, allele-specific primer extension, allele specific amplification, sequencing, 5′ nuclease digestion, molecular beacon assay, oligonucleotide ligation assay, size analysis, and single-stranded conformational polymorphism, can be used.

(3) Peripheral Biomarkers

At least one of serum/plasma/cerebrospinal fluid (CSF) and/or urine biomarkers as described herein can be measured according to methods known to one skilled in the art. In various embodiments, a blood sample (e.g., including a serum sample and/or a plasma sample) and/or a CSF sample and/or a urine sample can be collected from a subject and used to measure at least one biomarker as described herein.

Performing measurements of metabolites in a biological sample is known to a skilled artisan. For example, a target metabolite may optionally be separated (e.g., prior to detection) from a biological sample by gas chromatography (GC), e.g., when interfaced with mass spectrometry (GC-MS), and/or high performance liquid chromatography (HPLC), and/or capillary electrophoresis (CE). CE generally has higher theoretical separation efficiency than HPLC, and is suitable for use with a wider range of metabolite classes than is GC.

Levels of metabolites can be detected by any known methods in the art. For example, mass spectrometry (MS) can be used to identify and to quantify metabolites after separation by GC, HPLC (LC-MS), and/or CE. In some embodiments, MS can be used as a stand-alone technology, e.g., the biological sample is infused directly into the mass spectrometer which provides both separation and detection of metabolites.

1. A method of treating depression in a subject in need thereof, comprising administering a composition comprising S-adenosyl-L-methionine (SAMe) to the subject, wherein the subject has been identified as having one or more of the following biomarkers:
(a) a single nucleotide polymorphism (SNP) at position 677 of SEQ ID NO: 1 or position 27 of SEQ ID NO: 7 (identified by rs1801133) comprising at least one thymine (T) allele or the complement thereof, wherein the SEQ ID NO: 1 and SEQ ID NO: 7 are each independently a portion of a genomic nucleic acid sequence of methylenetetrahydrofolate reductase (MTHFR);
(b) a SNP at position 1298 of SEQ ID NO: 1 comprising at least one cytosine (C) allele or the complement thereof;
(c) a SNP at position 1793 of SEQ ID NO: 1 or position 27 of SEQ ID NO: 8 (identified by rs2274976) comprising at least one adenosine (A) allele or the complement thereof, wherein the SEQ ID NO: 1 and SEQ ID NO: 8 are each independently a portion of a genomic nucleic acid sequence of MTHFR;
(d) a SNP at position 2756 of SEQ ID NO: 2 or position 27 of SEQ ID NO: 9 (identified by rs1805087) comprising at least one guanosine (G) allele or the complement thereof, wherein the SEQ ID NO: 2 and SEQ ID NO: 9 are each independently a portion of a genomic nucleic acid sequence of methionine synthase (MTR);
(e) a SNP at position 66 of SEQ ID NO: 3 or position 27 of SEQ ID NO: 10 (identified by rs1801394) comprising at least one G allele or the complement thereof, wherein the SEQ ID NO: 3 and SEQ ID NO: 10 are each independently a portion of a genomic nucleic acid sequence of methionine synthase reductase (MTRR);
(f) a SNP at position 27 of SEQ ID NO: 11 (identified by rs1006737) comprising at least one A allele or the complement thereof, wherein the SEQ ID NO: 11 is a portion of a genomic nucleic acid sequence of calcium channel, voltage-dependent, L type, alpha 1C subunit (CACNA1C);
(g) a SNP at position 27 of SEQ ID NO: 12 (identified by rs1883729) comprising at least one A allele or the complement thereof, wherein the SEQ ID NO: 12 is a portion of a genomic nucleic acid sequence of DNA (cytosine-5)-methyltransferase 3 beta (DNMT3B);
(h) a SNP at position 27 of SEQ ID NO: 13 (identified by rs7163862) comprising at least one thymidine (T) allele or the complement thereof, wherein the SEQ ID NO: 13 is a portion of a genomic nucleic acid sequence of GTP cyclohydrolase 1 feedback regulatory protein (GCHFR);
(i) a SNP at position 27 of SEQ ID NO: 14 (identified by rs12659) comprising at least one T allele or the complement thereof, wherein the SEQ ID NO: 14 is a portion of a genomic nucleic acid sequence of reduced folate carrier protein (RCF2);
(j) a SNP at position 27 of SEQ ID NO: 15 (identified by rs202676) comprising at least one G allele or the complement thereof, wherein the SEQ ID NO: 15 is a portion of a genomic nucleic acid sequence of folate hydrolase (prostate-specific membrane antigen) 1 (FOLH1);
(k) a SNP at position 27 of SEQ ID NO: 16 (identified by rs2297291) comprising at least one A allele or the complement thereof, wherein the SEQ ID NO: 16 is a portion of a genomic nucleic acid sequence of reduced folate carrier protein (RCF1);
(l) a SNP at position 27 of SEQ ID NO: 17 (identified by rs1051266) comprising at least one A allele or the complement thereof, wherein the SEQ ID NO: 17 is a portion of a genomic nucleic acid sequence of reduced folate carrier protein (RCF1);
(m) a SNP at position 27 of SEQ ID NO: 18 (identified by rs8007267) comprising at least one T allele or the complement thereof, wherein the SEQ ID NO: 18 is a portion of a genomic nucleic acid sequence of GTP cyclohydrolase 1 (GCH1);
(n) a SNP at position 27 of SEQ ID NO: 19 (identified by rs7639752) comprising at least one A allele or the complement thereof, wherein the SEQ ID NO: 19 is a portion of a genomic nucleic acid sequence of choline-phosphate cytidylyltransferase A (PCYT1A);
(o) a SNP at position 27 of SEQ ID NO: 20 (identified by rs6275) comprising at least one T allele or the complement thereof, wherein the SEQ ID NO: 20 is a portion of a genomic nucleic acid sequence of dopamine receptor D2 (DRD2);
(p) a SNP at position 27 of SEQ ID NO: 21 (identified by rs1079596) comprising at least one T allele or the complement thereof, wherein the SEQ ID NO: 21 is a portion of a genomic nucleic acid sequence of dopamine receptor D2 (DRD2);
(q) a SNP at position 27 of SEQ ID NO: 22 (identified by rs11240594) comprising at least one A allele or the complement thereof, wherein the SEQ ID NO: 22 is a portion of a genomic nucleic acid sequence of dopamine receptor D2 (DRD2);
(r) a SNP at position 27 of SEQ ID NO: 23 (identified by rs4633) comprising at least one C allele or the complement thereof, wherein the SEQ ID NO: 23 is a portion of a genomic nucleic acid sequence of catechol-β-methyltransferase (COMT);
(s) a SNP at position 27 of SEQ ID NO: 24 (identified by rs4680) comprising at least one G allele or the complement thereof, wherein the SEQ ID NO: 24 is a portion of a genomic nucleic acid sequence of catechol-β-methyltransferase (COMT);
(t) a SNP at position 27 of SEQ ID NO: 25 (identified by rs250682) comprising at least one C allele or the complement thereof, wherein the SEQ ID NO: 25 is a portion of a genomic nucleic acid sequence of solute carrier family 6 (neurotransmitted transported, dopamine), member 3 (SLC6A3);
(u) a SNP at position 27 of SEQ ID NO: 26 (identified by rs2277820) comprising at least one T allele or the complement thereof, wherein the SEQ ID NO: 26 is a portion of a genomic nucleic acid sequence of formiminotransferase cyclodeaminase (FTCD);
(v) a SNP at position 27 of SEQ ID NO: 27 (identified by rs2236225) comprising at least one A allele or the complement thereof, wherein the SEQ ID NO: 27 is a portion of a genomic nucleic acid sequence of methylenetetrahydrofolate dehydrogenase (NADP+dependent) 1 (MTHFD1);
(w) an expression level ratio of S-adenosyl methionine (SAM) to S-adenosyl homocysteine (SAH) smaller than a pre-determined reference ratio;
(x) an expression level of 4-hydroxynonenal (4-FINE) greater than a first pre-determined reference value;
(y) an expression of high sensitive C-reactive protein (hsCRP) greater than a second pre-determined reference value;
(z) a homocysteine level greater than or equal to a third pre-determined reference value;
(aa) a folate level less than or equal to a fourth pre-determined reference value;
(bb) a B12 level less than a fifth pre-determined reference value;
(cc) a methylmalonic acid level greater than a sixth pre-determined reference value; and,
(dd) a formiminoglutamic acid level greater than or equal to a seventh pre-determined reference value.

2. The method of claim 1, wherein the SAMe comprises a gallic acid ester selected from the group consisting of methyl gallate, ethyl gallate, propyl gallate, butyl gallate, isobutyl gallate, isoamyl gallate, octyl gallate, dodecyl gallate, lauryl gallate, hexadecyl gallate, cetyl gallate, gallocatechol, gallocatechin, and epigallocatechin.

3. The method of claim 2, wherein the ratio (weight:weight) of gallic acid ester to SAMe ion is selected from the group consisting of: (a) from 5:1 to 1:400; (b) from 1:1 to 1:100; and, (c) from 1:2 to 1:80.

4. The method of claim 2, wherein the composition comprises from about 1 to about 400 mg of the gallic acid ester, from about 5 to about 200 mg of the gallic acid ester, or from about 5 to about 100 mg of the gallic acid ester.

5. The method of claim 2, wherein the composition comprises 0.1 to 40%, 0.1 to 25%, or 0.1 to 10% by weight of the gallic acid ester.

6. The method of claim 1, wherein the dose of SAMe ion is from about 50 mg to about 3200 mg.

7. The method of claim 1, wherein the composition comprises an oral dosage form.

8. The method of claim 7, wherein the oral dosage form comprises a functional coating selected from the group consisting of an enteric coating, time-release coating, and pH-dependent coating.

9. The method of claim 8, wherein the composition comprises a physiologically effective amount of SAMe, wherein non-parenteral administration of the composition to a selected human subject group produces in the selected human subject group an average SAMe plasma area under the curve (average AUC) of at least 600 ng·h/mL per each 100 mg dosage of SAMe ion delivered and an average C max of at least 110 ng/mL per each 100 mg dosage of SAMe ion delivered.

10. The method of claim 7, wherein the composition comprises 60-80% SAMe, 0-10% disintegrant, 0-1% colloidal silicon dioxide, 0.1-2% lubricant, and at least 7% of a binder, wherein all % values are based on the percentage by weight of the composition, and wherein oral administration of the composition to a selected human subject group produces in the subject group: (a) an average maximum SAMe blood plasma concentration (average C max) of at least about 110 ng/mL per each 100 mg of SAMe ion in the composition; and (b) an average SAMe plasma area under curve (average AUC) within a range of about 400 ng·h/mL to about 800 ng·h/mL per each 100 mg of SAMe ion in the composition.

11. The method of claim 10, wherein the disintegrant is selected from the group consisting of sodium starch glycolate, croscarmellose sodium, crosprovidone, and combinations thereof.

12. The method of claim 10, wherein the lubricant is selected from the group consisting of magnesium stearate, talc, calcium stearate, stearic acid, stearin hydrogenated vegetable oils, sodium benzoate, leucine, sodium stearyl fumarate, and combinations thereof.

13. The method of claim 10, wherein the binder is selected from the group consisting of a sugar, gelatin, a gum, microcrystalline cellulose, waxes, synthetic polymers, polyethylene glycol, polyvinyl pyrrolidone, and combinations thereof.

14. The method of claim 10, wherein the composition when administered to the selected human subject group provides in the subject group an average T max and/or C max with reduced variation.

15. The method of claim 10, wherein the composition when administered to the selected human subject group provides in the subject group an improved pharmacokinetic profile through: an equivalent average AUC to bi-daily dosing and/or reduced side effects through once a day dosing.

16. The method of claim 10, wherein oral administration of the composition to the selected subject group produces in the subject group an average C max of at least about 120 ng/mL per each 100 mg of SAMe ion in the composition.

17. The method of claim 1, wherein the depression is major depressive disorder.

18. The method of claim 1, wherein the subject is resistant to treatment with a selective serotonin reuptake inhibitor (SSRI).

19. The method of claim 1, wherein the subject has been identified as having one or more of:
(a) a SNP at position 27 of SEQ ID NO: 18 (rs8007267) comprising at least one T allele or the complement thereof; and,
(b) a SNP at position 27 of SEQ ID NO. 24 (rs4680) comprising two guanine G alleles or the complement thereof.

20. The method of claim 1, wherein the subject has been identified as having one or more of:
(a) a SNP at position 1298 of SEQ ID NO: 1 comprising at least one C allele or the complement thereof; and,
(b) a SNP at position 677 of SEQ ID NO: 1 or position 27 of SEQ ID NO: 27 comprising at least one T allele or the complement thereof.
